(19) United States
(12) Patent Application Publication
(10) Pub. No.: US 2025/0210134 A1
(43) Pub. Date: Jun. 26, 2025

(54) METHOD FOR EVALUATING CONSENSUS BASE ERROR RATE AND A SYSTEM THEREOF

(71) Applicant: Tsung-Lin Liu, Tainan (TW)

(72) Inventors: Tsung-Lin Liu, Tainan (TW); Shuen-Lin Jeng, Tainan (TW)

(21) Appl. No.: 19/039,364

(22) Filed: Jan. 28, 2025

Related U.S. Application Data
(63) Continuation of application No. PCT/CN2022/112752, filed on Aug. 16, 2022.

(51) Int. Cl. G16B 25/10; G06F 17/17

(52) U.S. Cl. CPC G16B 25/10; G06F 17/17

(57) ABSTRACT

The present invention discloses method for evaluating consensus base error rate and system thereof. The polymerase chain reaction error rate and the sequencing error rate are integrated to evaluate the error rate of individual consensus bases, and the quality scores of the consensus bases are calculated to improve the accuracy of existing molecular signature sequencing and eliminate subsequent interpretation difficulties caused by low-frequency mutation in mutation detection. Therefore, the method has the advantages of improving precise treatment in the future.

### CROSS-REFERENCE TO RELATED APPLICATION

This application is a continuation-in-part of PCT Application No. PCT/CN2022/112752, entitled CONCENSUS BASE ERROR RATE EVALUATION METHOD, filed on Aug. 16, 2022.

### FIELD OF THE INVENTION

The present invention is related to a technical field of DNA sequencing data analysis, and particularly related to an evaluation method of a consensus base error rate.

### BACKGROUND OF THE INVENTION

Detecting rare mutations is promising for personalized cancer diagnosis and treatment. While somatic variant calling guides the targeted therapy, detecting circulating tumor DNA serves as a non-invasive and cost-effective mean for characterizing cancer progression and monitoring the treatment response. Adopting these approaches, however, is challenging when the variant allele frequency (VAF) is comparable or even lower than the frequency of sequencing errors and other technical artifacts.

To address the challenge, molecular barcodes (MBCs) or unique molecular identifiers (UMIs) have been used and shown to effectively reduce sequencing errors. In UMI sequencing, each DNA template is first labeled by a unique UMI. The labeled templates are then amplified via polymerase chain reaction (PCR) and the resulting amplicons are randomly sampled for sequencing. By taking consensus of the sequenced amplicons, occasional sequencing errors can be eliminated.

However, PCR errors can be introduced during amplification, leading to error bases in a good portion of final amplicons when the errors arise in the first few cycles.

Such error bases can dominate in an UMI group especially when only few amplicons are sampled for sequencing (called small UMI size here). In real application, UMIs of a small size usually constitute majority of the data. When there are only 2 to 3 DNA sequences with the same UMI label, taking consensus may not eliminate PCR and/or sequencing errors if exist. Therefore, considering both PCR and sequencing errors is essential in the analysis of UMI sequencing data.

Several computational tools have been proposed to call variants using UMI data, e.g., DeepSNVMiner, smCounter, MAGERI, smCounter2, and UMI-VarCal. However, all these tools apply a heuristic approach to handle PCR errors. For example, smCounter estimates quality of a called variant using a joint probabilistic model of PCR and sequencing errors. The latter is calculated based on sequencing quality while a hand-waving heuristic approximation is used for the former. On the other hand, MAGERI only treats UMIs of size ≥5 and assumes all sequencing errors are eliminated in the consensus sequences. This approach discards majority of the data and the absence of sequencing errors cannot be fully guaranteed.

To handle PCR errors in the consensus sequences, MAGERI considers that error rates of a consensus base follow a beta distribution. Parameters of this phenomenological model are obtained via fitting to UMI training data of a known DNA template. This phenomenological model is later adopted in smCounter2. To utilize UMI data, smCounter2 builds a second beta-distribution model for UMIs of size <5, but that is still phenomenological and may not describe sequencing errors accurately. More importantly, these phenomenological models cannot be used to estimate quality of each UMI consensus base. Therefore, it is not appropriate to analyze UMI consensus sequences using conventional variant callers, as done by fgbio (http://fulcrumgenomics.github.io/fgbio/) or Mutect2 which requires quality information of each consensus base.

Additionally, the rule-based approach DeepSNVMiner is also unable to reveal PCR errors or sequencing errors, leading to low-confidence of the mutation detection. Besides, using UMI-VarCal to evaluate confidence of mutation detection is not accurate, as UMI-VarCal is also a rule-based method. The limitations of these approaches result from a lack of solid statistical framework to describe PCR and sequencing errors during UMI sequencing.

Chinese Pat. No. CN112687339B, entitled “Method and device for counting sequence errors in plasma DNA fragment sequencing data”, discloses a method and a device for counting sequence errors in plasma DNA fragment sequencing data.

Reading true UMI in a library building process, and taking the true UMI as a UMI reference sequence set; counting the times of reads UMI errors of all reads cycles in sequencing data and the proportion of error bases to current cycles bases; and counting the sequence error information of the reads of the complete testing template, and counting the error rate of each cycle by identifying the 5′ end UMI sequence and the 3′ end UMI sequence of the reads of the complete testing template. The cycle error rate counted by the method can be used for evaluating the data quality, and can also provide data correction bases and quality values of the bases in the subsequent clustering error correction process, so that prior probability is provided for clustering error correction and quality value correction, and the accuracy of low-frequency mutation detection is greatly improved; in this disclosure, PCR amplification error rate and sequencing error rate are integrated as a reference of base correction, but the error rate is determined post template sequencing, where sequence acquisition and alignment are conducted among distinct nucleic acid fragments. Such a method lacks any means to judge the true and false of the consensus base, resulting in difficulties of illustrating consensus base error rate and assigning quality score to each thereof. Moreover, such method is unattainable without UMI sequence library and sequence alignment.

### SUMMARY OF THE INVENTION

Low-frequency mutation detection is the foundation of precision medicine and personalized diagnosis, and development of this field will be profoundly influenced by evaluating the error rate of consensus bases especially when the mutation frequency is lower than sequencing error rate. With existing technologies, molecular barcodes are widely used to eliminate sequencing errors. Molecular barcodes with unique sequence are labeled to distinct nucleic acid fragments, and the DNA fragments carrying the molecular barcodes are subjected to PCR amplification prior to sequencing of the randomly sampled amplicons. Taking consensus of the amplicon sequences with the same UMI can eliminate sequencing errors in the amplicon sequences.

Nonetheless, certain PCR errors are inevitable during nucleic acid sequence amplification, which requires additional processing. Current computational tools are capable of handling molecular barcode data, but their evaluations of PCR error are based on empirical rules or phenomenological model, thus is unable to provide a quality score for each individual consensus base. This escalates uncertainty and inconvenience of mutant detection. Such limitation confronted by the current technologies results from a lack of a statistical framework to describe errors during PCR amplification and sequencing process simultaneously.

Accordingly, to provide a solution for issues of the current technologies, the present invention discloses a statistical framework for quantifying PCR errors and sequencing errors. The model facilitates quality evaluation of consensus bases using molecular barcodes, and assigns a quality score to each consensus base for determining confidence of the consensus base. The computational foundation of the aforementioned framework involves the nature of PCR process and sequencing quality information. The framework can also be applied to evaluate PCR error rate of each data set and provides a quality score for each consensus base.

Concretely, provided in the first aspect of the present invention is a method for evaluating consensus base error rate, comprising: selecting one or multiple amplified bases from an amplicon pool, wherein the amplicon pool is amplified from an unknown base N; sequencing a j^{th} base of the selected amplified bases to be an observed base sⱼ, and pooling the observed base sⱼ into an observed base collection S, wherein j is any one of positive integers; calculating a consensus base c(S) according to the observed base collection S; and calculating a consensus base error rate Pc(S), wherein the consensus base error rate Pc(S) is a probability that the consensus base c(S) is distinct from the unknown base N, and wherein: the consensus base error rate Pc(S) is a sum of probabilities that the unknown base N from which the observed base collection S is derived is identical to an assumed base b, but the assumed base b is distinct from the consensus base c(S), and wherein the assumed base b is selected from a group consisting of adenine (A), thymine (T), guanine (G) and cytosine (C), and the assumed base b is distinct from the consensus base c(S).

Preferably, the consensus base error rate Pc(S) is calculated through the following equation:

$${P}_{c ( S )} = P ( N \neq c ( S ) \vert S ) = {\sum }_{b \neq c ( S )} P ( N = b \vert S ) = \frac{{\sum }_{b \neq c ( S )} P ( S \vert N = b )}{{\sum }_{b = \{ A , C , G , T \}} P ( S \vert N = b )} ;$$

wherein, the P(S|N=b) is a probability of the observed base collection S being randomly selected from the amplicon pool if the unknown base N is identical to the assumed base b.

Preferably, the P(S|N=b) is calculated by a method comprising: defining a fraction of false bases F_{R}, wherein the fraction of false bases F_{R} is a ratio of an amplified false base to the amplified bases in the amplicon pool, and the amplified false base is a base distinct from the assumed base b; and calculating the P(S|N=b) through the following equation:

$$P ( S \vert N = b ) = {\sum }_{{F}_{R}} P ( {F}_{R} ) \cdot P ( S \vert N = {b∩F}_{R} = {F}_{R} ) ;$$

wherein the P(S|N=b ∩F_{R}=F_{R}) is a sum product of P(sⱼ|N=b ∩F_{R}=F_{R}) of each observed base sⱼ if the unknown base N is identical to the assumed base b and a ratio of the amplified false base in the amplicon pool is F_{R}.

More preferably, if the observed base sⱼ is identical to the assumed base b, the P(sⱼ|N=b ∩F_{R}=F_{R}) is equal to a fraction of true bases F_{R}′; if the observed base sⱼ is distinct from the assumed base b, the P(sⱼ|N=b ∩F_{R}=F_{R}) is equal to the fraction of false bases F_{R}, and wherein a sum of the fraction of true bases F_{R}′ and the fraction of false bases F_{R} is 1.

More preferably, the method further comprises: modifying the P(sⱼ|N=b ∩F_{R}=F_{R}) to a P(sⱼ=b′|N=b ∩{F_{b″≠b}}={F_{b″≠b}}) based on a sequencing accuracy rate P_{seq1}, a sequencing error rate P_{seq2} and an amplified base ratio F_{b″≠b}, wherein the P(sⱼ=b′|N=b ∩{F_{b″≠b}}={F_{b″≠b}}) is a probability that the observed base sⱼ is identical to another observed base b′ if the unknown base N is identical to the assumed base b and a ratio of one amplified base b″ distinct from the assumed base b in the amplicon pool is F_{b″≠b}, wherein: if the amplified base b″ is distinct from the assumed base b, the ratio of the amplified base b″ is equal to the fraction of false bases F_{R}, where the P(sⱼ=b′|N=b ∩{F_{b″≠b}}={F_{b″≠b}}) is modified to satisfy the following equation:

$$P ( {s}_{j} = {b}^{′} \vert N = b ∩ \{ {F}_{{b}^{″} \neq b} \} = \{ {F}_{{b}^{″} \neq b} \} ) = \{ \begin{matrix}{P}_{pcr 1} \times {P}_{seq 1} + {P}_{pcr 2} \times {P}_{seq 2} & {b}^{′} = b \\ {P}_{pcr 3} \times {P}_{seq 1} + {P}_{pcr 4} \times {P}_{seq 2} + {P}_{pcr 5} \times {P}_{seq 2} & {b}^{′} \neq b\end{matrix} ;$$

wherein, if the another observed base b′ is identical to the assumed base b, Pₚₑᵣ₁ represents a probability that the assumed base b is amplified into a base identical to the another observed base b′, and Pₚₑᵣ₂ represents a probability that the assumed base b is amplified into the amplified base b″, where the amplified base b″ is distinct from the assumed base b;
wherein, if the another observed base b′ is distinct from the assumed base b, Pₚₑᵣ₃ represents a probability that the assumed base b is amplified into a base identical to the another observed base b′, Pₚₑᵣ₄ represents a probability that the assumed base b is amplified into a base identical to the assumed base b, and Pₚₑᵣ₅ represents a probability that the assumed base b is amplified into the amplified base b″, where the amplified base b″ is distinct from the assumed base b and also distinct from the another observed base b′;
wherein, the sequencing accuracy rate P_{seq1} represents a probability that the another observed base b′ is correctly sequenced, and the sequencing error rate P_{seq2} represents a probability that the amplified base b″ is falsely sequenced as the another observed base b′.

More preferably, the method further comprises: calculating the sequencing accuracy rate P_{seq1} and the sequencing error rate P_{seq2} based on a sequencing quality score Qᵢ of the another observed base b′, wherein: the sequencing accuracy rate P_{seq1} is calculated through the following equation:

$${P}_{seq 1} = 1 - 1 {0}^{- {Q}_{i} / 10} ;$$

the sequencing error rate P_{seq2} is calculated through the following equation:

$${P}_{seq 2} = \frac{1 {0}^{- {Q}_{i} / 10}}{3} .$$

More preferably, the observed base collection S comprises d observed bases sⱼ, wherein d observed bases sⱼ comprise n_{b} bases identical to the assumed base b and (d-n_{b}) the amplified false bases distinct from the assumed base b, and wherein d is the maximum integer value of j, and n_{b} is a positive integer less than or equal to d;
if the observed base sⱼ is distinct from the assumed base b, the P(sⱼ|N=b ∩F_{R}=F_{R}) is equal to the fraction of false bases F_{R}, and a sum product of the P(sⱼ|N=b ∩F_{R}=F_{R}) is calculated through the following equation:

$${\prod }_{j = 1}^{d} P ( {s}_{j} \vert N = b ⋂ {F}_{R} = {F}_{R} ) = {( 1 - {F}_{R} )}^{{n}_{b}} {F}_{R}^{d - {n}_{b}} .$$

More preferably, the P(S|N=b) is obtained by moment approximation through the following equation:

$$P ( S \vert N = b ) = {\sum }_{k = 0}^{{n}_{b}} {{C}_{k}^{{n}_{b}} ( - 1 )}^{k} {M}_{( k + d - {n}_{b} )} ;$$

wherein the M_{(k+d-n}_{_{b}}₎ is a (k+d-n_{b})^{th} moment among a probability distribution of the fraction of false bases F_{R}, where k is any one of non-negative integer less than or equal to n_{b};
wherein an approximated value of the M_{(k+d-n}_{_{b}}⁾ is r/2^{k+d-nb}, where r represents a probability of polymerase chain reaction error occurrence at a gene locus corresponding to the unknown base N in each cycle.

Preferably, the method further comprises: retrieving a template sequence and labeling a molecular barcode thereto, and producing the amplicon pool by conducting a polymerase chain reaction based on the template sequence; and establishing the observed base collection S by sequencing bases randomly sampled from the amplicon pool, wherein the molecular barcode comprises a nucleic acid capture sequence or a unique molecular identifier sequence (UMI).

Provided in another aspect of the present invention is a system for evaluating consensus base error rate, comprising a polymerase chain reaction quality evaluation module configured to execute any one of the aforementioned methods so as to calculate a consensus base error rate P_{c(S)}.

Preferably, the system further comprises a sequencing quality evaluation module, being signally connected to the polymerase chain reaction quality evaluation module and configured to read a sequencing quality score Qᵢ of another observed base b′ so as to calculate a sequencing accuracy rate P_{seq1} and a sequencing error rate P_{seq2}, wherein:
the sequencing accuracy rate P_{seq1} is calculated through the following equation:

$${P}_{seq 1} = 1 - {10}^{- {Q}_{i} / 10} ;$$

the sequencing error rate P_{seq2} is calculated through the following equation:

$${P}_{seq 2} = \frac{{10}^{- {Q}_{i} / 10}}{3} ;$$

a consensus base error rate Pc(S) modifying module, configured in the polymerase chain reaction quality evaluation module and being signally connected to the sequencing quality evaluation module so as to modify the P(sⱼ|N=b ∩F_{R}=F_{R})to be P(sⱼ=b′|N=b ∩{F_{b″≠b}}={F_{b}″≠_{b}}) based on the sequencing accuracy rate P_{seq1}, the sequencing error rate P_{seq2} and an amplified base ratio F_{b″≠b}, where the P(sⱼ=b′|N=b ∩{F_{b″≠b}}={F_{b″≠b}}) represents a probability that the observed base sⱼ is identical to the another observed base b′ if the unknown base N is identical to the assumed base b, and a ratio of an amplified base b″ distinct from the assumed base b in the amplicon pool is F_{b″≠b}, and wherein:
if the amplified base b″ is distinct from the assumed base b, and the ratio of the amplified base F_{b″≠b} is equal to the fraction of amplified bases F_{R}, the P(sⱼ=b′|N=b ∩{F_{b″≠b}}={F_{b″≠b}}) is modified to satisfy the following equation:

$$P ( {s}_{j} = {b}^{′} \vert N = b ⋂ \{ {F}_{{b}^{″} \neq b} \} = \{ {F}_{{b}^{″} \neq b} \} ) = \{ \begin{matrix}{P}_{pcr 1} \times {P}_{seq 1} + {P}_{pcr 2} \times {P}_{seq 2} & {b}^{′} = b \\ {P}_{pcr 3} \times {P}_{seq 1} + {P}_{pcr 4} \times {P}_{seq 2} + {P}_{pcr 5} \times {P}_{seq 2} & {b}^{′} \neq b\end{matrix} ;$$

wherein, if the another observed base b′ is identical to the assumed base b, Pₚₑᵣ₁ represents a probability that the assumed base b is amplified into a base identical to the another observed base b′, and Pₚₑᵣ₂ represents a probability that the assumed base b is amplified into the amplified base b″, where the amplified base b″ is distinct from the assumed base b;
wherein, if the another observed base b′ is distinct from the assumed base b, Pₚₑᵣ₃ represents a probability that the assumed base b is amplified into the another observed base b′, Pₚₑᵣ₄ represents a probability that the assumed base b is amplified into a base identical to the assumed base b, and Pₚₑᵣ₅ represents a probability that the assumed base b is amplified into the amplified base b″, where the amplified base b″ is distinct from the assumed base b and also distinct from the another observed base b′;
wherein, the sequencing accuracy rate P_{seq1} represents a probability that the another observed base b′ is correctly sequenced, and the sequencing error rate P_{seq2} represents a probability that the amplified base b″ is falsely sequenced as the another observed base b′.

Provided in yet one another aspect of the present invention is a device for evaluating consensus base error rate, comprising a memory and a processor, wherein the memory is configured to store a program, and the processor is configured to execute the program so as to implement the aforementioned method for evaluating consensus base error rate.

The technical solutions provided in the present invention facilitate reflecting accurate error rates in detection of low-frequency mutation by calculating a quality score specific to each one of consensus bases. With regard of the quality score of the consensus base, expectedly, low-frequency mutation shall be detected with improved accuracy.

### BRIEF DESCRIPTION OF THE DRAWINGS

FIG. 1A is a flowchart illustrating the method provided in the first aspect of the present invention;

FIG. 1B is a flowchart illustrating the method provided in the second aspect of the present invention;

FIG. 1C is a probability distribution diagram illustrating the probability distribution of false error bases F₁₀ after a 10-cycle PCR amplification under PCR error rate of 0.01;

FIG. 2 is a probability distribution diagram illustrating the agreement of theoretical values and approximated values of error rates in different amplification cycle;

FIG. 3A is a block chart illustrating the system for evaluating consensus base error rate in the third aspect of the present invention;

FIG. 3B is a block chart illustrating the system for evaluating consensus base error rate in the fourth aspect of the present invention;

FIG. 4 is a block chart illustrating the device for evaluating consensus base error rate in the fifth aspect of the present invention; and

FIGS. 5A to 5B are probability distribution diagrams illustrating the agreement of observed and expected frequencies of consensus base quality in Example 4.

### DETAILED DESCRIPTION OF THE INVENTION

The following section provides a more detailed explanation of the present invention by combining specific embodiments with diagrams. The detailed descriptions in the embodiments aim to facilitate a better understanding of the invention. However, those skilled in the art will readily appreciate that certain technical features may be omitted in different circumstances or substituted with other components, materials, or methods. In some cases, certain operations related to the invention are not explicitly shown or described in the specification to avoid overshadowing the core aspects of the invention with excessive details. For those skilled in the art, a comprehensive understanding of these related operations can be readily achieved based on the descriptions in the specification and common technical knowledge in the field.

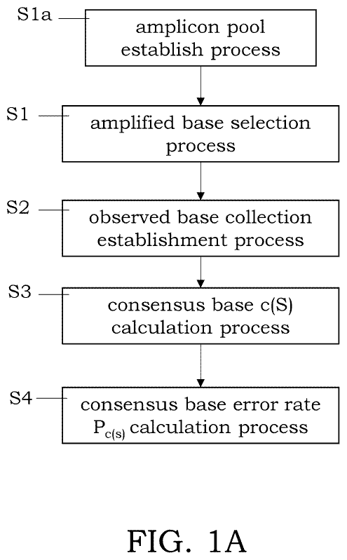

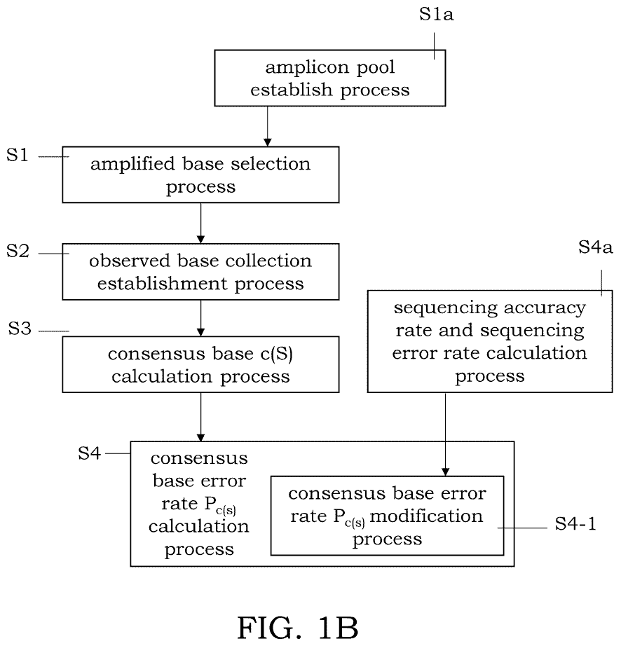

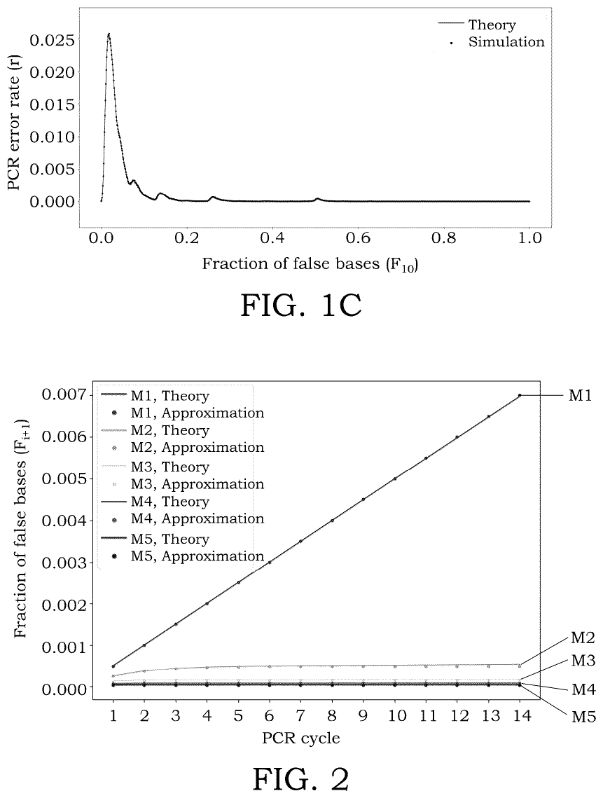

As shown in FIG. 1A, provided in the first aspect of the present invention is a method for evaluating consensus error rate, comprising:
amplified base selection process (S1): selecting one or multiple amplified bases from an amplicon pool, wherein the amplicon pool is amplified from an unknown base N;
observed base collection establishment process (S2): sequencing a j^{th} base of the selected amplified bases to be an observed base sⱼ, and pooling the observed base sⱼ into an observed base collection S, wherein j is any one of positive integers;
consensus base c(S) calculation process (S3): calculating a consensus base c(S) according to the observed base collection S; and
consensus base error rate P_{c(S)} calculation process (S4): calculating a consensus base error rate P_{c(S)}, wherein the consensus base error rate P_{c(S)} is a probability that the consensus base c(S) is distinct from the unknown base N, and wherein:
the consensus base error rate P_{c(S)} is a sum of probabilities that the unknown base N from which the observed base collection S is derived is identical to an assumed base b, but the assumed base b is distinct from the consensus base c(S), and wherein the assumed base b is selected from a group consisting of adenine (A), thymine (T), guanine (G) and cytosine (C), and the assumed base b is distinct from the consensus base c(S).

The term “consensus base c(S)” used herein refers to the observed base sⱼ with the highest occurrence frequency in the observed base collection S. For instance, in an observed base collection S having 6 observed base sⱼ, wherein the observed bases sⱼ are A, A, A, T, T and A, respectively, the consensus base c(S) in this observed base collection S is A whose occurrence frequency is ⅔ and larger than that of T. Accordingly, there are occurrences of non-consensus base c(S)′ among the amplified bases derived from the unknown base N. Regardless of sequencing error, the non-consensus base c(S)′ is caused by an error in polymerase chain reaction. Therefore, in the first aspect of the present invention, probability that the consensus base c(S) is not the assumed base, namely the error rate of consensus base c(S), is evaluated to serve as reference of judging confidence of the consensus base c(S).

In the consensus base error rate P_{c(S)} calculation process (S4), the consensus base error rate P_{c(S)} is calculated through the following equation:

$${P}_{c ( S )} = P ( N \neq c ( S ) \vert S ) = {\sum }_{b \neq c ( S )} P ( N = b \vert S ) = \frac{{\sum }_{b \neq c ( S )} P ( S \vert N = b )}{{\sum }_{b = \{ A , C , G , T \}} P ( S \vert N = b )} ;$$

wherein, the P(S|N=b) is a probability of the observed base collection S being randomly selected from the amplicon pool if the unknown base N is identical to the assumed base b.

In the consensus base error rate P_{c(S)} calculation process (S4), the P(S|N=b) is calculated by a method comprising: defining a fraction of false bases F_{R}, wherein the fraction of false bases F_{R} is a ratio of an amplified false base to the amplified bases in the amplicon pool, and the amplified false base is a base distinct from the assumed base b; and calculating the P(S|N=b) through the following equation:

$$P ( S \vert N = b ) = {\sum }_{{F}_{R}} P ( {F}_{R} ) \cdot P ( S \vert N = b ⋂ {F}_{R} = {F}_{R} ) ;$$

wherein the P(S|N=b ∩F_{R}=F_{R}) is a sum product of P(sⱼ|N=b ∩F_{R}=F_{R}) of each observed base sⱼ if the unknown base N is identical to the assumed base b and a ratio of the amplified false bases in the amplicon pool is F_{R}.

In various embodiments, if the observed base sⱼ is identical to the assumed base b, the P(sⱼ|N=b ∩F_{R}=F_{R}) is equal to a fraction of true bases F_{R}′; if the observed base sⱼ is distinct from the assumed base b, the P(sⱼ|N=b ∩F_{R}=F_{R}) is equal to the fraction of false bases F_{R}, and wherein a sum of the fraction of true bases F_{R}′ and the fraction of false bases F_{R} is 1.

In various embodiments, the observed base collection S comprises d observed bases sⱼ, wherein d observed bases sⱼ comprise n_{b} bases identical to the assumed base b and (d-n_{b}) the amplified false bases distinct from the assumed base b, and wherein d is the maximum integer value of j, and n_{b} is a positive integer less than or equal to d;
if the observed base sⱼ is distinct from the assumed base b, the P(sⱼ|N=b ∩F_{R}=F_{R}) is equal to the fraction of false bases F_{R}, and a sum product of the P(sⱼ|N=b ∩F_{R}=F_{R}) is calculated through the following equation:

$${\prod }_{j = 1}^{d} P ( {s}_{j} \vert N = b ⋂ {F}_{R} = {F}_{R} ) = {( 1 - {F}_{R} )}^{{n}_{b}} {F}_{R}^{d - {n}_{b}} .$$

In various embodiments, calculation of the P(S|N=b) is further expanded as followings:

$$\begin{matrix}P ( S \vert N = b ) = {\sum }_{{F}_{R}} P ( {F}_{R} ) \cdot P ( S \vert N = b ⋂ {F}_{R} = {F}_{R} ) \\ ≅ {\sum }_{{F}_{R}} P ( {F}_{R} ) \cdot {\prod }_{j = 1}^{d} P ( {s}_{j} \vert N = b ⋂ {F}_{R} = {F}_{R} ) \\ = {\sum }_{{F}_{R}} P ( {F}_{R} ) \cdot {( 1 - {F}_{R} )}^{{n}_{b}} \cdot {F}_{R}^{d - {n}_{b}}\end{matrix}$$

wherein P(F_{R})·F_{R}^{d-n}^{_{b}} represents the moment of a probability distribution of the fraction of false bases F_{R} in the observed base collection S. Since the amplicon pool is established by one or multiple cycles of amplification, calculation of the moment will be extremely intensive with a growing number of amplification cycles. Principally, a value of the moment P(F_{R})·F_{R}^{d-n}^{_{b}} can be calculated by approximation to reduce the intensity of calculations if only considering the first one amplification error. As such, approximation of the moment can be done as followings:

$$\begin{matrix}P ( S \vert N = b ) = {\sum }_{{F}_{R}} P ( {F}_{R} ) \cdot {( 1 - {F}_{R} )}^{{n}_{b}} \cdot {F}_{R}^{d - {n}_{b}} \\ = {\sum }_{{F}_{R}} P ( {F}_{R} ) \cdot {\sum }_{k = 0}^{{n}_{b}} {{C}_{k}^{{n}_{b}} ( - 1 )}^{k} {F}_{R}^{k + d - {n}_{b}} \\ = {\sum }_{k = 0}^{{n}_{b}} {{C}_{k}^{{n}_{b}} ( - 1 )}^{k} {M}_{( k + d - {n}_{b} )}\end{matrix} ;$$

wherein the M_{(k+d-n}_{_{b}}₎ is (k+d-n_{b})^{th} moment among a probability distribution of the fraction of false bases F_{R}, where k is any one of non-negative integer less than or equal to n_{b};
wherein an approximated value of the M_{(k+d-n}_{_{b}}₎ is r/2^{k+d-nb}, where r represents a probability of polymerase chain reaction error occurrence at a gene locus corresponding to the unknown base N in each cycle.

With reference to FIG. 1B, provided in the second aspect of the present invention is a method for evaluating consensus error rate P_{c(S)}. The steps and computational process of the method are overall the same as those in the first aspect, but the method includes sequencing error rates after amplification cycles are completed in addition to calculation of error rate during amplification cycles of the unknown base N.

To further elaborate, in an observed base collection S having 6 observed bases sⱼ representing A, A, A, T, T and A, the consensus base c(S) is A. The non-consensus base c(S) occurs in the observed base collection S, among the amplified bases derived from the unknown base N, may be a base distinct from the assumed base, such as T, or may be a base identical to the assumed base, such as A, being sequenced as T resulting from a sequencing error, or may be a base distinct from the assumed base and distinct from the non-consensus base C(S)′, such as C or G, being sequenced as T resulting from a sequence error. Considering such situations, a polymerase chain reaction error rate and a sequencing error rate are integrated into calculation of the consensus base error rate P_{c(S)} to serve as a reference of judging the confidence of the consensus base c(S).

Accordingly, in the second aspect of the present invention, the method further comprises:
consensus base error rate P_{c(S)} modification process (S4-1): modifying the P(sⱼ|N=b ∩F_{R}=F_{R}) to a P(sⱼ=b′|N=b ∩{F_{b″≠b}}={F_{b″≠b}}) based on a sequencing accuracy rate P_{seq1}, a sequencing error rate P_{seq2} and an amplified base ratio F_{b″≠b}, wherein the P(sⱼ=b′|N=b ∩{F_{b″≠b}}={F_{b″≠b}}) is a probability that the observed base sⱼ is identical to another observed base b′ if the unknown base N is identical to the assumed base b and a ratio of one amplified base b″ distinct from the assumed base b in the amplicon pool is F_{b″≠b}, wherein:
if the amplified base b″ is distinct from the assumed base b, the ratio of the amplified base b″ is equal to the fraction of false bases F_{R}, where the P(sⱼ=b′|N=b ∩{F_{b″≠b}}={F_{b″≠b}}) is modified to satisfy the following equation:

$$\begin{matrix}P ( {s}_{j} = {b}^{′} \vert N = b ⋂ \{ {F}_{{b}^{″} \neq b} \} = \{ {F}_{{b}^{″} \neq b} \} ) \\ = \{ \begin{matrix}{P}_{pcr 1} \times {P}_{seq 1} + {P}_{pcr 2} \times {P}_{seq 2} & {b}^{′} = b \\ \begin{matrix}{P}_{pcr 3} \times {P}_{seq 1} + {P}_{pcr 4} \times {P}_{seq 2} + \\ {P}_{pcr 5} \times {P}_{seq 2}\end{matrix} & {b}^{′} \neq b\end{matrix}\end{matrix} ;$$

wherein, if the another observed base b′ is identical to the assumed base b, Pₚₑᵣ₁ represents a probability that the assumed base b is amplified into a base identical to the another observed base b′, and Pₚₑᵣ₂ represents a probability that the assumed base b is amplified into the amplified base b″, where the amplified base b″ is distinct from the assumed base b;
wherein, if the another observed base b′ is distinct from the assumed base b, Pₚₑᵣ₃ represents a probability that the assumed base b is amplified into a base identical to the another observed base b′, Pₚₑᵣ₄ represents a probability that the assumed base b is amplified into a base identical to the assumed base b, and Pₚₑᵣ₅ represents a probability that the assumed base b is amplified into the amplified base b″, where the amplified base b″ is distinct from the assumed base b and also distinct from the another observed base b′;
wherein, the sequencing accuracy rate P_{seq1} represents a probability that the another observed base b′ is correctly sequenced, and the sequencing error rate P_{seq2} represents a probability that the amplified base b″ is falsely sequenced as the another observed base b′.

In preferred embodiments, the method further comprises: sequencing accuracy rate and sequencing error rate calculation process (S4a): calculating the sequencing accuracy rate P_{seq1} and the sequencing error rate P_{seq2} based on a sequencing quality score Qᵢ of the another observed base b′, wherein: the sequencing accuracy rate P_{seq1} is calculated through the following equation:

$${P}_{seq 1} = 1 - {10}^{- {Q}_{i} / 10} ;$$

the sequencing error rate P_{seq2} is calculated through the following equation:

$${P}_{seq 2} = \frac{{10}^{- {Q}_{i} / 10}}{3} .$$

In various embodiments, further with reference to FIGS. 1A to 1B, before selection of the amplified bases, the method further comprises an amplicon pool establish process (Sla), comprising:
retrieving a template sequence and ligating a molecular barcode thereto, and producing the amplicon pool by conducting a polymerase chain reaction based on the template sequence; and
establishing the observed base collection S by sequencing bases randomly sampled from the amplicon pool, wherein the molecular barcode comprises a nucleic acid capture sequence or a unique molecular identifier sequence (UMI), but not limited to this.

The following provides a further explanation of the specific derivations underlying the computational basis in the various embodiments of the present invention. First of all, it is assumed that after an ideal PCR cycle, each DNA template is duplicated, and the new copy has a sequence identical to the DNA template. While each nucleotide in the single-strand template remains intact, PCR error may occur at a locus in the new copy, resulting in a distinct base, thereby creating distinct bases in the subsequent PCR cycle.

Assumed that after R cycles, 2^{R} molecules (a.k.a. amplicons) are expected to arise from one double-strand DNA template, some may contain a base distinct from the one on the original template (called a false base). Fraction of the false bases depends on the PCR error rate r and the cycle number i at which the errors occur. From the 2^{R} amplicons, only a small portion (e.g., 1˜1,000 in 2²⁰⁻³⁰ amplicons) will be randomly selected for sequencing. When a sampled amplicon carries a false base, the PCR error can be observed. Therefore, observing PCR error can be imagined as a process of sampling amplicons that carry a false base from the 2^{R} amplicons, and the fraction of false bases F_{R} in the amplicon determines the frequency of PCR error. Thus, deriving a probability distribution of fraction of false bases F_{R} is required for PCR error modeling.

In the following, it is assumed that PCR errors at different loci are independent and only one locus is discussed. Let tᵢ and fᵢ be the numbers of true bases and false bases at the i^{th} cycle, Equation (I) describes the probability of having m and n additional false bases arising from the ti true bases and the f false bases in the next cycle (the (i+1)^{th} cycle), respectively. In Equation (I), r is the PCR error rate per base per cycle. Namely, r represents PCR error rate at a locus corresponding to the unknown base N (namely, the true base). On the other hand, the probability of m additional false bases arising from the tᵢ true bases simply follows a binomial distribution. For an additional false base to derive from the fᵢ false bases, it requires either no PCR error (with probability 1-r) or a PCR error that does not turn the false base back to the true one, which probability is r*⅔ assuming no base preference in PCR error. This explains the (1-r/3) term in Equation (I).

With Equation (I), the probability of having fᵢ₊₁ false bases at the (i+1)^{th} cycle can be calculated via considering all possible combinations of additional false bases arising from the false bases and true bases at the i^{th} cycle. In the equation (II), the Kronecker delta function δ ensures (_{f}_{ᵢ₊₁}_{-f}_{ᵢ}) additional false bases from the false bases and true bases at the it cycle. The number fᵢ can be 0, 1, 2, . . . and up to 2ⁱ−1 because at least the starting true base will persist through the duplications. The upper bound is further limited by _{f}_{ᵢ₊₁} because _{f}_{ᵢ}_{≤f}_{ᵢ₊₁}, i.e., false bases remain false in the next cycle.

$${P}_{add} ( m , n ; {t}_{i} , {f}_{i} ) = {{C}_{m}^{{t}_{i}} ( r )}^{m} {( 1 - r )}^{{t}_{i} - m} \cdot {{C}_{n}^{{f}_{i}} ( 1 - \frac{r}{3} )}^{n} {( \frac{r}{3} )}^{{f}_{i} - n} \qquad ( I )$$

$$P ( {f}_{i + 1} ) = {\sum }_{{f}_{i} = 0}^{min ( {2}^{i} - 1 , {f}_{i + 1} )} P ( {f}_{i} ) {\sum }_{m = 0}^{{t}_{i}} {\sum }_{n = 0}^{{f}_{i}} δ ( m + n , {f}_{i + 1} - {f}_{i} ) {P}_{add} ( m , n ; {t}_{i} , {f}_{i} ) \qquad ( II )$$

With the above dynamic equation, the probability distribution of P(F_{R}) can be obtained via calculation of P(fᵢ₊₁). For example, shown in FIG. 1C is the probability distribution of F₁₀ under a PCR error rate 0.01. As shown in FIG. 1C, three minor peaks appear near fractions 0.5, 0.25, and 0.125. These peaks correspond to a first PCR error at the 1^{st} 2^{nd} and 3^{rd} cycle, resulting in about ½, ¼, and ⅛ of the bases being false, respectively. In addition to mathematical induction, random PCR errors may be simulated as well. In one example, with 1 million times of mathematical simulation with a PCR error rate 0.01 in a 10-cycle PCR, the resulting distribution of fractions of false bases agree well with that of mathematical induction.

With the probability distribution of F_{R}, a probability that a consensus base is not the true base may be calculated assuming no sequencing error. At one specific locus, let a sequenced base sᵢ be the base on the i^{th} sampled amplicon and S={sᵢ} be the collection of all bases. The term “consensus base c(S)” refers to the most frequent base in the observed base collection S. Probability P(N≠c(S)|S) that consensus base c(S) is not the unknown base N can be calculated via a Bayesian approach in Equation (III).

$$\begin{matrix}P ( N \neq c ( S ) \vert S ) = {\sum }_{b \neq c ( S )} P ( N = b \vert S ) \\ = \frac{{\sum }_{b \neq c ( S )} P ( S \vert N = b ) \cdot P ( N = b )}{{\sum }_{b = \{ A , C , G , T \}} P ( S \vert N = b ) \cdot P ( N = b )}\end{matrix} \qquad ( III )$$

Assuming no prior knowledge on the unknown base N, which means that probability of the unknown base N being any one of A, T, C, G is the same, P(N=b) will be a constant ¼. Thus, the terms P(N=b) can be crossed out from the equation (III). P(S|N=b) is the probability of obtaining observed base collection S via random sampling from 2^{R} amplicons arising from an assumed base b. The probability P(S|N=b) depends on the fraction of false bases F_{R} in the amplicons and can be calculated via considering all possible fraction of false bases F_{R} values as in Equation (IV). Given an F_{R}, the probability that a randomly sampled base is false is simply F_{R} as illustrated in Equation (V).

$$\begin{matrix}P ( S \vert N = b ) = {\sum }_{{F}_{R}} P ( {F}_{R} ) \cdot P ( S \vert N = b ⋂ {F}_{R} = {F}_{R} ) \\ ≅ {\sum }_{{F}_{R}} P ( {F}_{R} ) \cdot {\prod }_{i} P ( {s}_{i} \vert N = b ⋂ {F}_{R} = {F}_{R} )\end{matrix} \qquad ( IV )$$

$$P ( {s}_{i} \vert N = b ⋂ {F}_{R} = {F}_{R} ) = \{ \begin{matrix}1 - {F}_{R} & {s}_{i} = b \\ {F}_{R} & {s}_{i} \neq b\end{matrix} \qquad ( V )$$

When multiple amplicons are sampled, the above formulae involve the calculation of ΣP(F_{R})*F_{R}^{k}·ΣP(F_{R})*F_{R}^{k} refers to the k^{th} moment of the fraction of false bases F_{R} probability distribution. In principle, the numerical values can be obtained using Equation (II). However, the calculation of ΣP(F_{R})*F_{R}^{k} is too intense to be handled practically for a large R. Therefore, an approximation of these moments is needed. The moments can be approximated via considering only the first PCR error when the error rate is relatively small. For example, in a PCR of 10 cycles, there are 2¹⁰−1=1,023 duplication events. Under a PCR error rate 0.001, the mean number of PCR errors is about 1.

Besides, a PCR error occurring at an earlier cycle results in a higher fraction of false bases in the final amplicons, and thus contributes more to the moments. When only the first PCR error is considered, the moments can be calculated as in Equation (VI). In the equation (VI), i stands for the cycle at which the first PCR error occurs. For a relatively small PCR error rate r (i.e., r<2^{-R}), probability that the first PCR error occurs at the i^{th} cycle is approximately 2ⁱ⁻¹*r because there are 2ⁱ⁻¹ amplicons before the i^{th} cycle and the chance of PCR error on each amplicon is r.

The resulting fraction of false bases F_{R} is (½ⁱ) without further PCR errors. Notwithstanding, further PCR errors indeed may occur, but their contributions to moments belong to higher order terms (i.e., r², r³, . . . ), and the moments are much smaller when r<<1. FIG. 2 shows theoretical values of the moments of fraction of false bases F_{R} at different PCR cycles. As shown in FIG. 2, theoretical values of the first 5 moments of fraction of false bases F_{R} at different cycles under a PCR error rate 0.001 may be calculated based on Equation (II). Meanwhile, FIG. 2 also shows the approximated moments based on Equation (VI), and these moments agree well with the theoretical values. With the approximated moments, probability that a consensus base c(S) is not the unknown base N (true base) can be calculated efficiently without the detailed probability distribution of fraction of false bases F_{R}. This is the key simplification in PCR error modelling.

$${M}_{k} ( R , r ) = {\sum }_{i = 1}^{R} {2}^{i - 1} {r ( \frac{1}{{2}^{i}} )}^{k} = \frac{r}{2} \cdot {\sum }_{i = 1}^{R} {2}^{- ( k - 1 ) i} = \frac{r}{2} \cdot \frac{( 1 - {2}^{- ( k - 1 ) R} )}{( {2}^{k - 1} - 1 )} {\to }^{R ≫ 1} \frac{r}{2} \cdot \frac{1}{( {2}^{k - 1} - 1 )} {\to }^{k ≫ 1} \frac{r}{{2}^{k}} \qquad ( VI )$$

With the moments, probability that the consensus base c(S) is not the unknown base N (true base) is calculated explicitly in Equations (VII) and (VIII). For instance, two types of observed bases sⱼ are assumed. Without loss of generality, let the observed bases sⱼ be A's and C's, and in the observed base collection S, the number n_{A} of A's is larger than n_{C} of C's, thus the consensus base c(S) is A. In Equation (VIII), when PCR error rate r<<1, probabilities P(S|N=G) and P(S|N=T) of the unknown base N not being G or T are neglected, because their magnitudes are much smaller than the probabilities P(S|N=A) and P(S|N=C) of the unknown base N being A or C. It is worth of notion, Equations (VII) and (VIII) should be modified if a different number of base types is observed and the modifications are shown later.

$$P ( S \vert N = b ) ≅ {\sum }_{{F}_{R}} P ( {F}_{R} ) \cdot {\prod }_{i = 1}^{d} P ( {s}_{i} \vert N = b ⋂ {F}_{R} = {F}_{R} ) = {\sum }_{{F}_{R}} P ( {F}_{R} ) \cdot {( 1 - {F}_{R} )}^{{n}_{b}} \cdot {F}_{R}^{d - {n}_{b}} = {\sum }_{{F}_{R}} P ( {F}_{R} ) \cdot {\sum }_{k = 0}^{{n}_{b}} {{C}_{k}^{{n}_{b}} ( - 1 )}^{k} {F}_{R}^{k + d - {n}_{b}} = {\sum }_{k = 0}^{{n}_{b}} {{C}_{k}^{{n}_{b}} ( - 1 )}^{k} {M}_{( k + d - {n}_{b} )} \qquad ( VII )$$

$$\begin{matrix}P ( N \neq A \vert S ) = \begin{matrix}\frac{{\sum }_{b = \{ C , G , T \}} ( S \vert N = b )}{{\sum }_{b = \{ A , C , G , T \}} ( S \vert N = b )} ≅ \\ \frac{P ( S \vert N = C )}{P ( S \vert N = A ) + P ( S \vert N = C )}\end{matrix} \\ = \frac{{\sum }_{k = 0}^{{n}_{C}} {{C}_{k}^{{n}_{C}} ( - 1 )}^{k} {M}_{( k + {n}_{c} )}}{{\sum }_{k = 0}^{{n}_{A}} {{C}_{k}^{{n}_{A}} ( - 1 )}^{k} {M}_{( k + {n}_{A} ) 00} + {\sum }_{k = 0}^{{n}_{C}} {{C}_{k}^{{n}_{C}} ( - 1 )}^{k} {M}_{( k + {n}_{c} )}}\end{matrix} \qquad ( VIII )$$

In addition to PCR error, sequencing error can result in a false observed bases sⱼ. Therefore, sequencing error is also integrated into evaluation of probability that the consensus base c(S) is not the unknown base N. In general terms, probability qᵢ that the sequenced base sᵢ is an error can be estimated by the quality score Qᵢ as in Equation (IX). If there is no preference of false bases in sequencing error, the probability of observing one of the false bases is qᵢ/3. With such information, PCR error and sequencing error for estimating quality of a consensus base c(S) can be integrated.

$${q}_{i} = {10}^{- {Q}_{i} / 10} = P ( {s}_{i} \neq N ) = P ( {s}_{i} \neq b \vert N = b ) \qquad ( IX )$$

To consider both PCR error and sequencing error, the particular details of estimating probability that consensus base c(S) is not the unknown base N shall be further elaborated on Equation (IV). Specifically, P(sᵢ|N=b∩F_{R}=F_{R}) shall be rewritten. Equation (X) describes the probability of observing another observed base b′ when the unknown base N is the assumed base b, and F_{b″≠b} stands for a fraction of false bases of another type of amplified base b″ in the final amplicon pool and represents the set of the amplified bases b″ when placed in “{ }”. When another observed base b′ is the assumed base b, it can be the result of no PCR error (P̅E̅) nor sequencing error (S̅E̅), or the result of a PCR error giving rise to a false amplified base b″ (PE_{bb″≠b}) and a sequencing error that turns the amplified base b″ back to the assumed base b (SE_{b″b}).

When the another observed base b′ is not the assumed base b, there are three possible schemes: (1) a PCR error giving rise to the another observed base b′ distinct from the assumed base b (PE_{bb′}) and no sequencing error, (2) no PCR error but a sequencing error that turns the assumed base b into the another observed base b′ (SE_{bb′}), and (3) a PCR error giving rise to a false amplified base b″ that is not the assumed base b nor the another observed base b′ (PE_{bb″≠{b,b′}}) and a sequencing error that turns the amplified base b″ into the another observed base b′ (SE_{b″ b′}).

Assuming independence between PCR error and sequencing error, probabilities of the above schemes are shown explicitly in Equation (X). In the equation (X), F_{R} stands for the total fraction of false bases, F_{R}=Σ_{b″≠b}F_{b″} for instance, which matches the earlier definition. Combining Equation (X) into Equations (VII) and (VIII), a quality score of the consensus base c(S) can be calculated and given to while taking into account both PCR error and sequencing error.

$$P ( {s}_{i} = {b}^{′} \vert N = b ⋂ \{ {F}_{{b}^{″} \neq b} \} = \{ {F}_{{b}^{″} \neq b} \} ) = \{ \begin{matrix}P ( \overline{PE} ⋂ \overline{SE} ) + P ( {PE}_{{bb}^{″} \neq b} ⋂ {SE}_{{b}^{″} b} ) & {b}^{′} = b \\ P ( {PE}_{{bb}^{′}} ⋂ \overline{SE} ) + P ( \overline{PE} ⋂ {SE}_{{bb}^{′}} ) + P ( {PE}_{{bb}^{″} \neq \{ b , {b}^{′} \}} ⋂ {SE}_{{b}^{″} b} ) & {b}^{′} \neq b\end{matrix} = \{ \begin{matrix}( 1 - {F}_{R} ) ( 1 - {q}_{i} ) + {F}_{R} \cdot \frac{{q}_{i}}{3} & {b}^{′} = b \\ {F}_{{b}^{′}} ( 1 - {q}_{i} ) + ( 1 - {F}_{R} ) \cdot \frac{{q}_{i}}{3} + {\sum }_{{b}^{″} \neq \{ b , {b}^{′} \}} {F}_{b}^{″} \cdot \frac{{q}_{i}}{3} & {b}^{′} \neq b\end{matrix} = \{ \begin{matrix}( 1 - {q}_{i} ) + ( \frac{4 {q}_{i}}{3} - 1 ) {F}_{R} & {b}^{′} = b \\ \frac{{q}_{i}}{3} + ( 1 - \frac{4 {q}_{i}}{3} ) {F}_{{b}^{′}} & {b}^{′} \neq b\end{matrix} \qquad ( X )$$

In the above formulation, three types of false observed bases are lumped together. The formulation needs further modification because the three types of false observed bases do not appear equally likely. For example, given A as the assumed base b, it is more likely to observe two C's in the final amplicon pool than one C and one G because the former can be achieved with only one PCR error while the latter requires at least two PCR errors.

Therefore, all four types of bases shall be considered in the formulation. Specifically, instead of using the probability distribution P(F_{R}) to modify the formulation, the four base version P({F_{C}, F_{G}, F_{T}}) will substitute of P(F_{R}) when the assumed base b is A. It should be noted that the key simplification of the PCR error modeling is approximation of moments. With four types of bases, approximation of the multivariate moments defined in Equation (XI) is required. Again, the moments can be approximated via considering only the first few PCR errors when r is much smaller than 1.

$${M}_{x y z} ( R , r ) = {\sum }_{\{ {F}_{C} , {F}_{G} , {F}_{T} \}} P ( \{ {F}_{C} , {F}_{G} , {F}_{T} \} ) {F}_{C}^{x} {F}_{G}^{y} {F}_{T}^{z} ≡ {\sum }_{\{ {F}_{{b}^{″} \neq A} \}} P ( \{ {F}_{{b}^{″} \neq A} \} ) {F}_{C}^{x} {F}_{G}^{y} {F}_{T}^{z} \qquad ( XI )$$

The evaluation model can also be extended to consider the double-strand nature of DNA. For a double-strand template, a PCR error produces an error base on the synthesized strand, and it takes one more cycle for the error base to settle on both strands. Therefore, a PCR error at the 1^{st} cycle leads to false bases in a quarter instead of half of the final amplicon pool. To further elaborate, a UMI usually labels only one strand of a DNA template. With these features, the multivariate moments can be approximated. On this occasion, the level of a multivariate moment is defined as the number of zeros in the orders (i.e., x, y, and z). A higher-level moment is smaller.

The above examples are intended merely to explain mathematical induction of the statistical model of the method for evaluating consensus base error rate in the present invention, but not intended to limit practical implementation of the method.

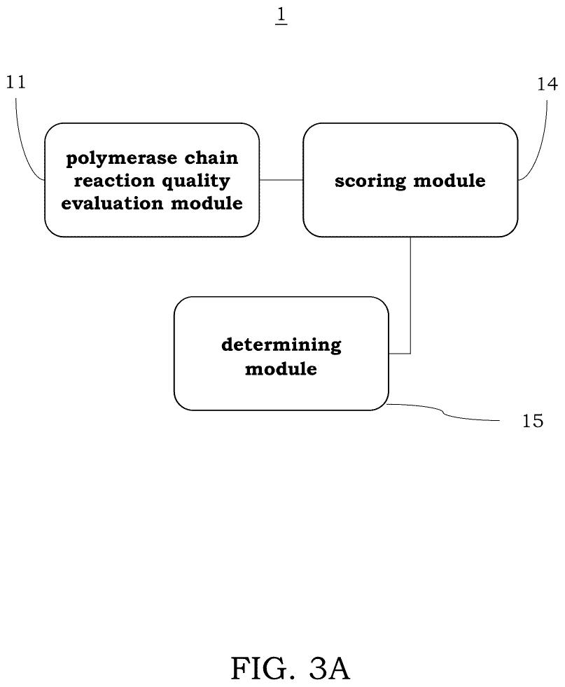

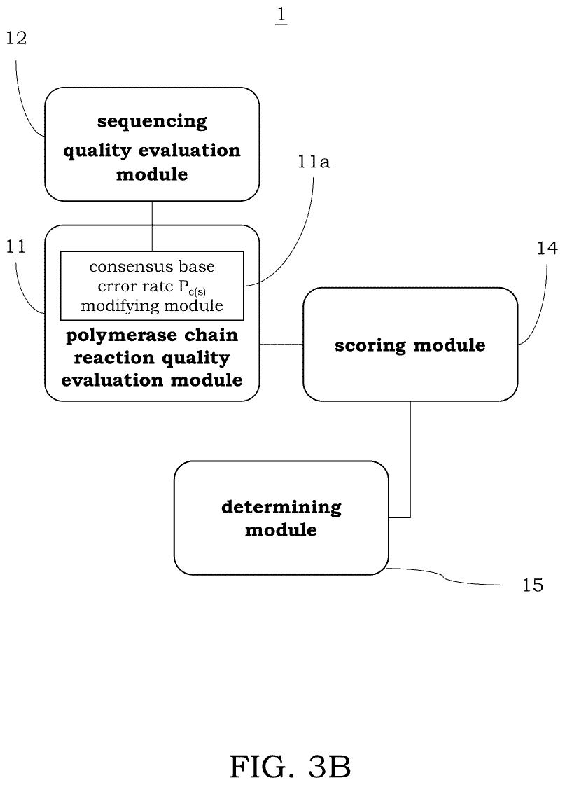

Provided in third aspect of the present invention is a system (1) for evaluating consensus base error rate. As shown in FIG. 3A, the system (1) comprises a polymerase chain reaction quality evaluation module (11) configured to execute the method in the first aspect of the present invention so as to calculate a consensus base error rate P_{c(S)}.

In various embodiments, as shown in FIG. 3A, the system (1) further comprises a scoring module (14), being signally connected to the polymerase chain reaction quality evaluation module (11), configured to identify the consensus base error rate P_{c(S)} and assign the consensus base c(S) a quality score Q_{c}.

In other embodiments, as shown in FIG. 3A, the system (1) further comprises a determining module (15), being signally connected to the scoring module (14) and the polymerase chain reaction quality evaluation module (11), configured to receive the quality score Q_{c} and compare the quality score Q_{c} with a second quality score P′_{c(S)} so as to generate a determined instruct, wherein the second quality score P′_{c(S)} is output by the polymerase chain reaction quality evaluation module (11), where:
if the second quality score P′_{c(S)} larger than the quality score Q_{c}, the determined instruct presents that the consensus base c(S) is confident;
if the second quality score P′_{c(S)} smaller than the quality score Q_{c}, the determined instruct presents that the consensus base c(S) is not confident.

Exemplarily, when a user obtains the quality score Q_{c}, the user may focus on the base set with high quality scores, and sets a cutoff score Qₓ taking the quality score Q_{c} as a baseline. In the subsequent batch processing of the sequencing data, confidence of the consensus base c(S) may be determined according to the cutoff score Qₓ. In the aforementioned case, the cutoff score setting may be extended to a consensus sequence consisting of one or multiple consensus bases c(S). Another example is provided herein to explain determination of a consensus sequence's confidence. Table 1 demonstrates a consensus sequence 1 and a consensus sequence 2 obtained from different batches of sequencing data resulting from an unknown sequence. The system (1) identifies that the consensus sequence 1 is 5′-CAA-3′ according to observed base collections including S₁₋₁={A₁C₁C₁}, S₁₋₂={A₂G₂A₂} and S₁₋₃={G₃A₃A₃} read from 5′ end to 3′ end. On the other hand, the consensus sequence 2 is determined to be 5′-CAG-3′ according to observed base collections including S₂₋₁={A₁C₁C₁}, S₂₋₂={A₂G₂A₂} and S₂₋₃={G₃G₃A₃}. After calculation of quality score of each consensus base, the system (1) identifies that the quality score Q_{c} of consensus base A obtained from the observed base collection S₁₋₃ is smaller than the cutoff score Qₓ, thereby giving a determined instruct of non-confidence. In contrast, all consensus bases in the observed base collections S₂₋₁, S₂₋₂ and S₂₋₃ have quality scores Q_{c} larger than the cutoff score Qₓ, and thus the system (1) gives a determined instruct of confidence. In this context, the user is capable of determining the consensus sequence confidence based on the determined instructs provided by the system (1) so that the most appropriate sequence reading can be made.

TABLE 1

| Consensus | | Observed | | Consensus | Quality | Determined |
|---|---|---|---|---|---|---|
| sequence | | base collection | | base | score (Qc) | instruct |
| 1 | 5′-CAA-3′ | S₁₋₁ | A₁C₁C₁ | C | >Qₓ | confident |
| | | S₁₋₂ | A₂G₂A₂ | A | >Qₓ | confident |
| | | S₁₋₃ | G₃A₃A₃ | A | <Qₓ | Not confident |
| 2 | 5′-CAG-3′ | S₂₋₁ | A₁C₁C₁ | C | >Qₓ | confident |
| | | S₂₋₂ | A₂G₂A₂ | A | >Qₓ | confident |
| | | S₂₋₃ | G₃G₃A₃ | G | >Qₓ | confident |

Provided in the fourth aspect of the present invention is a system or evaluating consensus base error rate. As shown in FIG. 3B, the basic elements and processing principles are the same as those in third aspect of the present invention, but the system (1) further comprises a sequencing quality evaluation module (12), being signally connected to the polymerase chain reaction quality evaluation module (11), configured to read a sequencing quality score Qᵢ of another observed base b′ so as to calculate a sequencing accuracy rate P_{seq1} and a sequencing error rate P_{seq2}, wherein:

the sequencing accuracy rate P_{seq1} is calculated through the following equation:

$${P}_{seq 1} = 1 - 1 {0}^{- {Q}_{i} / 10} ;$$

the sequencing error rate P_{seq2} is calculated through the following equation:

$${P}_{s e q 2} = \frac{{10}^{- {Q}_{i} / 10}}{3} ;$$

a consensus base error rate P_{c(S)} modifying module (11a), being configured in the polymerase chain reaction quality evaluation module (11) and signally connected to the sequencing quality evaluation module so as to modify the P(sⱼ|N=b ∩F_{R}=F_{R})to be P(sⱼ=b′|N=b ∩{F_{b″≠b}}={F_{b″≠b}}) based on the sequencing accuracy rate P_{seq1}, the sequencing error rate P_{seq2} and an amplified base ratio F_{b″≠b}, where the P(sⱼ=b′|N=b ∩{F_{b″≠b}}={F_{b″≠b}}) represents a probability that the observed base sⱼ is identical to the another observed base b′ if the unknown base N is identical to the assumed base b, and a ratio of a amplified base b″ distinct from the assumed base b in the amplicon pool is F_{b}*_{b}, and wherein:
if the amplified base b″ is distinct from the assumed base b, and the ratio of the amplified base F_{b″≠b} is equal to the fraction of amplified bases F_{R}, the P(sⱼ=b′|N=b ∩{F_{b″≠b}}={F_{b″≠b}}) is modified to satisfy the following equation:

$$P ( {s}_{j} = {b}^{′} \vert N = b ⋂ \{ {F}_{{b}^{″} \neq b} \} = \{ {F}_{{b}^{″} \neq b} \} ) = \{ \begin{matrix}{P}_{p c r 1} \times {P}_{s e q 1} + {P}_{p c r 2} \times {P}_{s e q 2} & {b}^{′} = b \\ {P}_{p c r 3} \times {P}_{s e q 1} + {P}_{p c r 4} \times {P}_{s e q 2} + {P}_{p c r 5} \times {P}_{s e q 2} & {b}^{′} \neq b\end{matrix} ;$$

wherein, if the another observed base b′ is identical to the assumed base b, Pₚₑᵣ₁ represents a probability that the assumed base b is amplified into a base identical to the another observed base b′, and Pₚₑᵣ₂ represents a probability that the assumed base b is amplified into the amplified base b″, where the amplified base b″ is distinct from the assumed base b;
wherein, if the another observed base b′ is distinct from the assumed base b, Pₚₑᵣ₃ represents a probability that the assumed base b is amplified into the another observed base b′, Pₚₑᵣ₄ represents a probability that the assumed base b is amplified into a base identical to the assumed base b, and Pₚₑᵣ₅ represents a probability that the assumed base b is amplified into the amplified base b″, where the amplified base b″ is distinct from the assumed base b and also distinct from the another observed base b′;
wherein, the sequencing accuracy rate P_{seq1} represents a probability that the another observed base b′ is correctly sequenced, and the sequencing error rate P_{seq2} represents a probability that the amplified base b″ is falsely sequenced as the another observed base b′.

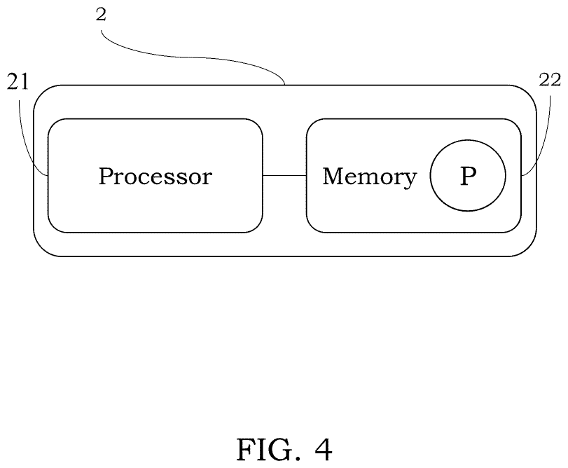

Please refer to FIG. 4, provided in fifth aspect of the present invention is a device (2) for evaluating consensus base error rate, comprising a memory (21) and a processor (22), wherein the memory (21) is configured to store a program (P), and the processor (22) is configured to execute the program (P) so as to realize the method for evaluating consensus base error rate in the first aspect or the second aspect of the present invention, or to realize the system in third aspect or the fourth aspect of the present invention.

The entirety or part of the functions of the method provided by the present invention can be implemented through hardware integration or software programming. When all or part of the functions are implemented via software, the program can be stored in a memory, which may include a read-only memory (ROM), random-access memory (RAM), magnetic disk, optical disk, hard disk, or other storage media. The processor executes the program to realize the aforementioned functions. For example, the program can be stored in the memory of a sequencing device, and by executing the program through the processor, all or part of the aforementioned functions can be achieved. Additionally, the program may also be stored in a server, another computer, a magnetic disk, an optical disk, flash memory, or a portable hard drive. Through downloading, transmission, copying, or other methods, the program can be transferred to the memory of the sequencing device or used to overwrite or update different versions of the sequencing system. Once the processor executes the program stored in the memory, all or part of the functions of the above method can be implemented.

### Example 1: Calculating Consensus Base c(S) Quality Considering Both PCR and Sequencing Errors

Example 1 illustrates a calculation process of probability that a consensus base c(S) is not a true base using the aforesaid method. An observed base collection includes three sequenced bases S={A₁C₂A₃} and the consensus base c(S) is determined to be A. The following equation (XII) shows probability of observing each base given a known assumed base b and composition of the false bases {F_{b″≠b}} based on Equation (X). In equation (XII), the uₖᵢ and vₖᵢ are defined to simplify notations and are used when the observed base is identical to and distinct from the assumed base b, respectively. The two indices k and i indicate order (i.e., terms associated with F^{k}) and the i^{th} observed base, respectively. With Equation (XII), probabilities of observing an observed base collection S given an assumed base b can be calculated with Equation (XIII).

The calculations can be expressed in terms of weighted multivariate moments. The weights are determined by the following factors together, including whether the observed bases match the assumed base b (i.e., u or v), orders of the moments, and sequencing qualities of the observed bases (i.e., qᵢ's). A factor “3” appears before some moments because the corresponding “F” term is purely F_{R} representing the total fraction of 3 types of false bases. With these weights, probability that a consensus base c(S) is not the assumed base b can be calculated in a more tractable manner with Equation (XIV). In Example 1, exactly two types of bases are present. When there are three types of observed bases, the calculations can be modified following a similar logic.

$$P ( {A}_{1 , 3} \vert N = A ⋂ \{ {F}_{{b}^{″} \neq A} \} = \{ {F}_{{b}^{″} \neq A} \} ) = ( 1 - {q}_{1 , 3} ) + ( \frac{4 {q}_{1 , 3}}{3} - 1 ) ( {F}_{C} + {F}_{G} + {F}_{T} ) ≡ {u}_{0} ( {q}_{1 , 3} ) + {u}_{1} ( {q}_{1 , 3} ) {F}_{R} ≡ {u}_{0 ( 1 , 3 )} + {u}_{1 ( 1 , 3 )} {F}_{R} \\ P ( {C}_{2} \vert N = A ⋂ \{ {F}_{{b}^{″} \neq A} \} = \{ {F}_{{b}^{″} \neq A} \} ) = \frac{{q}_{2}}{3} + ( 1 - \frac{4 {q}_{2}}{3} ) {F}_{C} ≡ v ( {q}_{2} ) + {v}_{1} ( {q}_{2} ) {F}_{C} ≡ {v}_{02} + {v}_{12} {F}_{C} \\ P ( {A}_{1 , 3} \vert N = C ⋂ \{ {F}_{{b}^{″} \neq C} \} = \{ {F}_{{b}^{″} \neq C} \} ) = {v}_{0 ( 1 , 3 )} + {v}_{1 ( 1 , 3 )} {F}_{A} P ( {C}_{2} \vert N = C ⋂ \{ {F}_{{b}^{″} \neq C} \} = \{ {F}_{{b}^{″} \neq C} \} ) = {u}_{02} + {u}_{12} {F}_{R} P ( {A}_{1 , 3} \vert N = G , T ⋂ \{ {F}_{{b}^{″} \neq G , T} \} = \{ {F}_{{b}^{″} \neq G , T} \} ) = {v}_{0 ( 1 , 3 )} + {v}_{1 ( 1 , 3 )} {F}_{A} P ( {C}_{2} \vert N = G , T ⋂ \{ {F}_{{b}^{″} \neq G , T} \} = \{ {F}_{{b}^{″} \neq G , T} \} ) = {v}_{02} + {v}_{12} {F}_{C} \qquad ( XII )$$

$$P ( S \vert N = A ) ≅ {\sum }_{\{ {F}_{{b}^{″} \neq A} \}} P ( \{ {F}_{{b}^{″} \neq A} \} ) \cdot {\prod }_{i = 1}^{3} P ( {s}_{i} \vert N = A ⋂ \{ {F}_{{b}^{″} \neq A} \} = \{ {F}_{{b}^{″} \neq A} \} ) = {\sum }_{\{ {F}_{{b}^{″} \neq A} \}} P ( \{ {F}_{{b}^{″} \neq A} \} ) \cdot ( {u}_{01} + {u}_{11} {F}_{R} ) ( {v}_{02} + {v}_{12} {F}_{C} ) ( {u}_{03} + {u}_{13} {F}_{R} ) = {\sum }_{\{ {F}_{{b}^{″} \neq A} \}} P ( \{ {F}_{{b}^{″} \neq A} \} ) \cdot [ {u}_{01} {v}_{02} {u}_{03} + ( {u}_{11} {v}_{02} {u}_{03} + {u}_{01} {v}_{02} {u}_{13} ) {F}_{R} + {u}_{11} {v}_{02} {u}_{13} {F}_{R}^{2} ] + {\sum }_{\{ {F}_{{b}^{″} \neq A} \}} P ( \{ {F}_{{b}^{″} \neq A} \} ) \cdot [ \text{} {u}_{01} {v}_{12} {u}_{03} {F}_{C} + ( {u}_{11} {v}_{12} {u}_{03} + {u}_{01} {v}_{12} {u}_{13} ) {F}_{R} {F}_{C} + {u}_{11} {v}_{12} {u}_{13} {F}_{R}^{2} {F}_{C} ] = {u}_{01} {v}_{02} {u}_{03} + ( {u}_{11} {v}_{02} {u}_{03} + {u}_{01} {v}_{02} {u}_{13} ) ( {M}_{100} + {M}_{010} + {M}_{001} ) + {u}_{11} {v}_{02} {u}_{03} ( {M}_{200} + {M}_{020} + {M}_{002} + 2 {M}_{110} + 2 {M}_{101} + 2 {M}_{011} ) + {u}_{01} {v}_{12} {u}_{03} \cdot {M}_{100} + ( {u}_{11} {v}_{12} {u}_{03} + {u}_{01} {v}_{12} {u}_{13} ) ( {M}_{200} + {M}_{110} + {M}_{101} ) + {u}_{11} {v}_{12} {u}_{13} ( {M}_{300} + {M}_{120} + {M}_{102} + 2 {M}_{210} + 2 {M}_{201} + 2 {M}_{111} ) ≅ {u}_{01} {v}_{02} {u}_{03} + ( {u}_{11} {v}_{02} {u}_{03} + {u}_{01} {v}_{02} {u}_{13} ) \cdot 3 {M}_{100} + {u}_{11} {v}_{02} {u}_{03} \cdot 3 {M}_{200} + {u}_{01} {v}_{12} {u}_{03} \cdot {M}_{100} + ( {u}_{11} {v}_{12} {u}_{03} + {u}_{01} {v}_{12} {u}_{13} ) {M}_{200} + {u}_{11} {v}_{12} {u}_{13} {M}_{300} = {u}_{01} {v}_{02} {u}_{03} + ( 3 {u}_{11} {v}_{02} {u}_{03} + {u}_{01} {v}_{12} {u}_{03} + 3 {u}_{01} {v}_{02} {u}_{13} ) {M}_{100} + ( {u}_{11} {v}_{12} {u}_{03} + 3 {u}_{11} {v}_{02} {u}_{13} + {u}_{01} {v}_{12} {u}_{13} ) {M}_{200} + {u}_{11} {v}_{12} {u}_{13} {M}_{300} ≡ {a}_{0} \cdot {M}_{000} + {a}_{1} \cdot {M}_{100} + {a}_{2} \cdot {M}_{200} + {a}_{3} \cdot {M}_{300} = {\sum }_{k = 0}^{3} {a}_{k} \cdot {M}_{k 00} \\ P ( S \vert N = C ) ≅ {\sum }_{\{ {F}_{{b}^{″} \neq C} \}} P ( \{ {F}_{{b}^{″} \neq C} \} ) \cdot ( {v}_{01} + {v}_{11} {F}_{A} ) ( {u}_{02} + {u}_{12} {F}_{R} ) ( {v}_{03} + {v}_{13} {F}_{A} ) ≅ {v}_{01} {u}_{02} {v}_{03} + ( {v}_{11} {u}_{02} {v}_{03} + 3 {v}_{01} {u}_{12} {v}_{03} + {v}_{01} {u}_{02} {v}_{13} ) {M}_{100} + ( {v}_{11} {u}_{12} {v}_{03} + {v}_{11} {u}_{02} {v}_{13} + {v}_{01} {u}_{12} {v}_{13} ) {M}_{200} + {v}_{11} {u}_{12} {v}_{13} {M}_{300} ≡ {\sum }_{k = 0}^{3} {c}_{k} \cdot {M}_{k 00} \\ P ( S \vert N = G , T ) ≅ {\sum }_{\{ {F}_{{b}^{″} \neq G , T} \}} P ( \{ {F}_{{b}^{″} \neq G , T} \} ) \cdot ( {v}_{01} + {v}_{11} {F}_{A} ) ( {v}_{02} + {v}_{12} {F}_{C} ) ( {v}_{03} + {v}_{13} {F}_{A} ) = {v}_{01} {v}_{02} {v}_{03} + ( {v}_{11} {v}_{02} {v}_{03} + {v}_{01} {v}_{12} {v}_{03} + {v}_{01} {v}_{02} {v}_{13} ) {M}_{100} + {v}_{11} {v}_{02} {v}_{13} {M}_{200} + ( {v}_{01} {v}_{12} {v}_{13} + {v}_{11} {v}_{12} {v}_{03} ) {M}_{110} + {v}_{11} {v}_{12} {v}_{13} {M}_{210} ≅ {v}_{01} {v}_{02} {v}_{03} + ( {v}_{11} {v}_{02} {v}_{03} + {v}_{01} {v}_{12} {v}_{03} + {v}_{01} {v}_{02} {v}_{13} ) {M}_{100} + {v}_{11} {v}_{02} {v}_{13} {M}_{200} ≡ {\sum }_{k = 0}^{3} {g}_{k} , {t}_{k} \cdot {M}_{k 00} \qquad ( XIII )$$

$$P ( N \neq A \vert S = \{ {A}_{1} {C}_{2} {A}_{3} \} ) = \frac{P ( S \vert N = C ) + P ( S \vert N = G ) + P ( S \vert N = T )}{P ( S \vert N = A ) + P ( S \vert N = C ) + P ( S \vert N = G ) + P ( S \vert N = T )} = \frac{{\sum }_{k = 0}^{3} ( {c}_{k} + {g}_{k} + {t}_{k} ) \cdot {M}_{k 00} ( R , r )}{{\sum }_{k = 0}^{3} ( {a}_{k} + {c}_{k} + {g}_{k} + {t}_{k} ) \cdot {M}_{k 00} ( R , r )} \qquad ( XIV )$$

### Example 2: Estimating PCR Error Rates

Example 2 takes adenine (A) as the assumed base b, when one type of false bases (C, G, or T) is present, the first level moment Mₓ₀₀'s in Equation (XI) can be modified via replacing the (r/3) by the corresponding substitution rate, such as p_{AC}, p_{AG}, or P_{AT}. As shown in Equation (XV), mₓ₀₀ is defined as the part of moment without substitution rate, thereby denoted by p_{bb′}. Similarly, the second level moments can be modified via replacing the (r/3)² by products of the corresponding substitution rates, such as p_{AC}*p_{AG} for M_{xy0}'s when the false base types are C and G as shown in Equation (XVI). To calculate quality of a consensus base c(S), one can simply replace the single-variate moments in the original formulation with the multivariate moments.

$${M}_{x 00}^{b {b}^{″}} ( R ) = {P}_{{bb}^{″}} {\sum }_{i = 1}^{R} {2}^{i - 1} {( \frac{1}{{2}^{i}} )}^{x} ≡ {P}_{{bb}^{″}} \cdot {m}_{x 00} ( R ) {\to }^{R ≫ 1 \& x ≫ 1} {p}_{b {b}^{″}} \cdot \frac{1}{{2}^{x}} \qquad ( XV )$$

$${M}_{xy 0}^{b {b}^{′} {b}^{″}} ( R ) ≡ {p}_{{bb}^{′}} {p}_{{bb}^{″}} \cdot {m}_{xy 0} ( R ) {\to }^{R ≫ 1} \{ \begin{matrix}{p}_{{bb}^{′}} {p}_{{bb}^{″}} ( \frac{{R}^{2}}{4} - 1 ) & x = y = 1 \\ {p}_{{bb}^{′}} {p}_{{bb}^{″}} ( {c}_{x 10 R} R + {c}_{x 10 c} ) & x > 1 \& y = 1 \\ {p}_{{bb}^{′}} {p}_{{bb}^{″}} ( {c}_{xy 10 c} ) & x > 1 \& y > 1\end{matrix} \qquad ( XVI )$$

Practically, these PCR substitution rates can be estimated using a maximal likelihood approach when the assumed bases b are known. In Example 2, let's focus on the loci where the template base is A. With an assumption of no sequencing error, a non-A base at the locus indicates a PCR error.

In Example 2, the loci with two or three types of false bases are neglected, as their frequency is much smaller under a low PCR error rate. Given observed bases at the loci, likelihood of the observation is described in Equation (XVII), where independence between loci is assumed. In the following equations, Sᵢ is the observed bases at locus i, {Sᵢ} stands for the observed base collection, and {p_{bb″}} represents the PCR error evaluation model described in the present invention. Calculation of the likelihood further requires the probability that all observed bases are A's as shown in Equation (XVIII), and the probability that the observed base collection includes some false bases, such as cytosines (C's) as shown in Equation (XIX).

$$L ( \{ {S}_{i} \} \vert N = A \& \{ {p}_{{bb}^{″}} \} ) = {\prod }_{i} P ( {S}_{i} \vert N = A \& \{ {p}_{{bb}^{″}} \} ) \qquad ( XVIII )$$

$$P ( {S}_{i} = \{ {A}_{1} … {A}_{{a}_{i}} \} \vert N = A \& \{ {p}_{{bb}^{″}} \} ) ≅ 1 + {\sum }_{k = 1}^{{a}_{i}} {{C}_{k}^{{a}_{i}} ( - 1 )}^{k} ( {p}_{A C} + {p}_{A G} + {p}_{A T} ) \cdot {m}_{k 00} ≡ 1 + a {m}_{i} \cdot ( {p}_{A C} + {p}_{A G} + {p}_{A T} ) \qquad ( XVIII )$$

$$P ( {S}_{i} = \{ {A}_{1} … {A}_{{a}_{i}} {C}_{1} … {C}_{{c}_{i}} \} \vert N = A \& \{ {p}_{{bb}^{″}} \} ) ≅ {\sum }_{k = 0}^{{a}_{i}} {{C}_{k}^{{a}_{i}} ( - 1 )}^{k} {p}_{A C} \cdot {m}_{( {a}_{i} + {c}_{i} ) 00} ≡ c {m}_{i} \cdot {p}_{A C} \qquad ( XIX )$$

In Example 2, a maximal likelihood approach is applied to estimate the substitution rate p_{AC}, and p_{AC} gives a zero derivative of the log-likelihood. In Equation (XX), n_{AA} and n_{AC} are numbers of loci with all bases as A and containing only A's and C's, respectively. Based on the Equation (XX), p_{AC} is estimated to be proportional to number of loci at which the A is turned into a C, wherein the denominator involves moments for loci without a false base, which reflects the PCR process.

$$\frac{d log L}{{dp}_{A C}} = {\sum }_{i = 1}^{{n}_{A A}} \frac{d}{{dp}_{A C}} log P ( {S}_{i} \vert N = A \& \{ {p}_{{bb}^{″}} \} ) + {\sum }_{j = 1}^{{n}_{A C}} \frac{d}{{dp}_{A C}} log P ( {S}_{j} \vert N = A \& \{ {p}_{{bb}^{″}} \} ) = 0 \qquad ( XX )$$

$${\sum }_{i = 1}^{{n}_{A A}} \frac{d}{{dp}_{A C}} log ( 1 + a {m}_{i} \cdot ( {p}_{A C} + {p}_{A G} + {p}_{A T} ) ) + {\sum }_{j = 1}^{{n}_{A C}} \frac{d}{{dp}_{A C}} log ( c {m}_{i} \cdot {p}_{A C} ) = 0$$

$${\to }^{\{ {p}_{{bb}^{″}} \} ≪ 1} {\sum }_{i = 1}^{{n}_{A A}} \frac{d}{{dp}_{A C}} a {m}_{i} \cdot ( {p}_{A C} + {p}_{AG} + {p}_{A T} ) + {\sum }_{j = 1}^{{n}_{A C}} \frac{1}{{p}_{A C}} = 0$$

$${\sum }_{i = 1}^{{n}_{A A}} a {m}_{i} + \frac{{n}_{A C}}{{p}_{A C}} = 0 \to {p}_{A C} = \frac{{n}_{A C}}{- {\sum }_{i = 1}^{{n}_{A A}} a {m}_{i}} = \frac{{n}_{A C}}{{\sum }_{i = 1}^{{n}_{A A}} ( {\sum }_{k = 1}^{{a}_{i}} {{C}_{k}^{{a}_{i}} ( - 1 )}^{k + 1} {m}_{k 00} )}$$

### Example 3: Estimating Background Errors

Once error probabilities of UMI all consensus bases are calculated, one can set a cutoff score and focus only on the high-quality bases. Among the high-quality bases, it is fair to assume only few false bases due to PCR and/or sequencing errors. Therefore, most of the remaining false bases can be attributed to background errors, for example, spontaneous mutation during DNA fragmentation. By applying UMI sequencing on known DNA templates, background error rate in the UMI sequencing can be estimated using the consensus base error rate P_{c(S)} obtained in Example 1.

### Example 4

UMI sequencing of random sequences was simulated to examine validity of consensus qualities derived from the PCR and sequencing error model as described hereinabove. One hundred random sequences of length 100 bp were generated as the targeted reference sequences of “normal cells”. Totally 10⁷ loci in 1000 normal cells were subject to mutation detection. Each targeted reference sequence of each normal cell was attached with a UMI label, and the UMI-labeled double-strand sequences were subject to 20-cycle PCR amplification, where the error rate r was 0.001.

For each UMI, three or five of the amplicons were randomly sampled for simulated Illumina sequencing. Sequenced amplicons of each UMI were assembled into a consensus sequence and quality of each consensus base was calculated using the method for evaluating consensus base error rate as described hereinabove. In Example 4, false consensus bases were identified and the frequency for each quality score was also calculated. The observed frequency was then compared to the expected value (e.g., 10⁻⁴ for Q=40).

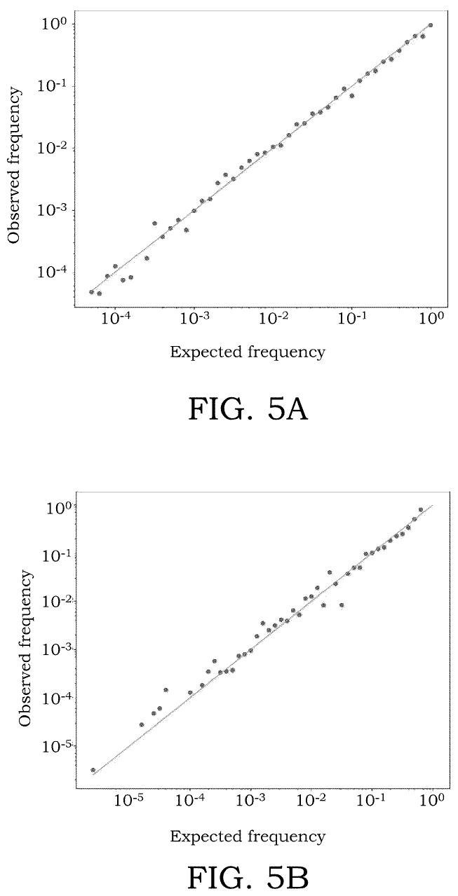

FIG. 5A shows a good agreement between the observed and expected frequencies across all consensus qualities with a UMI size three. As shown in FIG. 5B, for UMI size five, the overall agreement was still good except for the slightly larger fluctuations for some quality scores because there were not enough loci with such scores and/or only few or even no false consensus bases as the error rate was too low. The model fitting in Example 4 demonstrates that the method provided in the present invention was able to inform the true error probabilities of consensus bases (P_{c(S)}), and the error probability can be the base of quality scores calculation of a consensus base or even a consensus sequence.

The present invention provides a method for evaluating consensus base error rate based on a consensus base error rate evaluation model incorporating PCR error and sequencing error, and accurate evaluation of the consensus base error rate in UMI sequencing can be achieved using the aforesaid method.

Moreover, by using the method, each consensus base in a consensus sequence can be assigned of a precise quality score, which eliminates the impacts of low-frequency mutation on sequencing accuracy, thereby enhancing sequencing precision, and providing developmental foundation of precision medicine in the future.

What is claimed is:

1. A method for evaluating consensus error rate, comprising:
selecting one or multiple amplified bases from an amplicon pool, wherein the amplicon pool is amplified from an unknown base N;
sequencing a j^{th} base of the selected amplified bases to be an observed base sⱼ, and pooling the observed base sⱼ into an observed base collection S, wherein j is any one of positive integers;
calculating a consensus base c(S) according to the observed base collection S; and
calculating a consensus base error rate P_{c(S)}, wherein the consensus base error rate P_{c(S)} is a probability that the consensus base c(S) is distinct from the unknown base N, and wherein:
the consensus base error rate P_{c(S)} is a sum of probabilities that the unknown base N from which the observed base collection S is derived is identical to an assumed base b, but the assumed base b is distinct from the consensus base c(S), and wherein the assumed base b is selected from a group consisting of adenine (A), thymine (T), guanine (G) and cytosine (C), and the assumed base b is distinct from the consensus base c(S).

2. The method according to claim 1, wherein the consensus base error rate P_{c(S)} is calculated through the following equation:
$${P}_{c ( S )} = P ( N \neq c ( S ) \vert S ) = {\sum }_{b \neq c ( S )} P ( N = b \vert S ) = \frac{{\sum }_{b \neq c ( S )} P ( S \vert N = b )}{{\sum }_{b = \{ A , C , G , T \}} P ( S \vert N = b )} ;$$
wherein, the P(S|N=b) is a probability of the observed base collection S being randomly selected from the amplicon pool if the unknown base N is identical to the assumed base b.

3. The method according to claim 2, wherein the P(S|N=b) is calculated by a method comprising:
defining a fraction of false bases F_{R}, wherein the fraction of false bases F_{R} is a ratio of an amplified false base to the amplified bases in the amplicon pool, and the amplified false base is a base distinct from the assumed base b; and
calculating the P(S|N=b) through the following equation:
$$P ( S \vert N = b ) = {\sum }_{{F}_{R}} P ( {F}_{R} ) \cdot P ( S \vert N = b ⋂ {F}_{R} = {F}_{R} ) ;$$
wherein the P(S|N=b ∩F_{R}=F_{R}) is a sum product of P(sⱼ|N=b ∩F_{R}=F_{R}) of each observed base sⱼ if the unknown base N is identical to the assumed base b and a ratio of the amplified false base in the amplicon pool is F_{R}.

4. The method according to claim 3, wherein:
if the observed base sⱼ is identical to the assumed base b, the P(sⱼ|N=b ∩F_{R}=F_{R}) is equal to a fraction of true bases F_{R}′;
if the observed base sⱼ is distinct from the assumed base b, the P(sⱼ|N=b ∩F_{R}=F_{R}) is equal to the fraction of false bases F_{R}, and wherein a sum of the fraction of true bases F_{R}′ and the fraction of false bases F_{R} is 1.

5. The method according to claim 4, further comprising:
modifying the P(sⱼ|N=b ∩F_{R}=F_{R}) to a P(sⱼ=b′|N=b ∩{F_{b″≠b}}={F_{b″≠b}}) based on a sequencing accuracy rate P_{seq1}, a sequencing error rate P_{seq2} and an amplified base ratio F_{b″≠b}, wherein the P(sⱼ=b′|N=b ∩{F_{b″≠b}}={F_{b″≠b}}) is a probability that the observed base sⱼ is identical to another observed base b′ if the unknown base N is identical to the assumed base b and a ratio of one amplified base b″ distinct from the assumed base b in the amplicon pool is F_{b″≠b}, wherein:
if the amplified base b″ is distinct from the assumed base b, the ratio of the amplified base b″ is equal to the fraction of false bases F_{R}, where the P(sⱼ=b′|N=b ∩{F_{b″≠b}}={F_{b″≠b}}) is modified to satisfy the following equation:
$$P ( {s}_{j} = {b}^{′} \vert N = b ∩ \{ {F}_{{b}^{″} \neq b} \} = \{ {F}_{{b}^{″} \neq b} \} ) = \{ \begin{matrix}{P}_{pcr 1} \times {P}_{seq 1} + {P}_{pcr 2} \times {P}_{seq 2} & {b}^{′} = b \\ {P}_{pcr 3} \times {P}_{seq 1} + {P}_{pcr 4} \times {P}_{seq 2} + {P}_{pcr 5} \times {P}_{seq 2} & {b}^{′} \neq b\end{matrix} ;$$
wherein, if the another observed base b′ is identical to the assumed base b, Pₚₑᵣ₁ represents a probability that the assumed base b is amplified into a base identical to the another observed base b′, and Pₚₑᵣ₂ represents a probability that the assumed base b is amplified into the amplified base b″, where the amplified base b″ is distinct from the assumed base b;
wherein, if the another observed base b′ is distinct from the assumed base b, Pₚₑᵣ₃ represents a probability that the assumed base b is amplified into a base identical to the another observed base b′, Pₚₑᵣ₄ represents a probability that the assumed base b is amplified into a base identical to the assumed base b, and Pₚₑᵣ₅ represents a probability that the assumed base b is amplified into the amplified base b″, where the amplified base b″ is distinct from the assumed base b and also distinct from the another observed base b′;
wherein, the sequencing accuracy rate P_{seq1} represents a probability that the another observed base b′ is correctly sequenced, and the sequencing error rate P_{seq2} represents a probability that the amplified base b″ is falsely sequenced as the another observed base b′.

6. The method according to claim 5, further comprising:
calculating the sequencing accuracy rate P_{seq1} and the sequencing error rate P_{seq2} based on a sequencing quality score Qᵢ of the another observed base b′, wherein:
the sequencing accuracy rate P_{seq1} is calculated through the following equation:
$${P}_{seq 1} = 1 - 1 {0}^{- {Q}_{i} / 10} ;$$
the sequencing error rate P_{seq2} is calculated through the following equation:
$${P}_{seq 2} = \frac{1 {0}^{- {Q}_{i} / 10}}{3} .$$

7. The method according to any one of claim 4, wherein the observed base collection S comprises d observed bases sⱼ, wherein d observed bases sⱼ comprise n_{b} bases identical to the assumed base b and (d-nb) the amplified false bases distinct from the assumed base b, and wherein the d is the maximum integer value of j, and n_{b} is a positive integer less than or equal to d;
if the observed base sⱼ is distinct from the assumed base b, the P(sⱼ|N=b ∩F_{R}=F_{R}) is equal to the fraction of false bases F_{R}, and a sum product of the P(sⱼ|N=b ∩F_{R}=F_{R}) is calculated through the following equation:
$${\prod }_{j = 1}^{d} P ( {s}_{j} \vert N = b ∩ {F}_{R} = {F}_{R} ) = {( 1 - {F}_{R} )}^{{n}_{b}} {F}_{R}^{d - {n}_{b}} .$$

8. The method according to claim 7, wherein the P(S|N=b) is obtained by moment approximation through the following equation:
$$P ( S \vert N = b ) = {\sum }_{k = 0}^{{n}_{b}} {{C}_{k}^{{n}_{b}} ( - 1 )}^{k} {M}_{( k + d - {n}_{b} )} ;$$
wherein the M_{(k+d-n}_{_{b}}₎ is a (k+d-n_{b})^{th} moment among a probability distribution of the fraction of false bases F_{R}, where k is any one of non-negative integer less than or equal to n_{b};
wherein an approximated value of the M_{(k+d-n}_{_{b}}₎ is r/2^{k+d-nb}, where r represents a probability of polymerase chain reaction error occurrence at a gene locus corresponding to the unknown base N in each cycle.

9. A system for evaluating consensus base error rate, comprising:
a polymerase chain reaction quality evaluation module configured to execute a method according to claim 1 so as to calculate a consensus base error rate P_{c(S)}.

10. The system according to claim 9, wherein the consensus base error rate P_{c(S)} is calculated through the following equation:
$${P}_{c ( S )} = P ( N \neq c ( S ) \vert S ) = {\sum }_{b \neq c ( S )} P ( N = b \vert S ) = \frac{{\sum }_{b \neq c ( S )} P ( S \vert N = b )}{{\sum }_{b = \{ A , C , G , T \}} P ( S \vert N = b )} ;$$
wherein, the P(S|N=b) is a probability of the observed base collection S being randomly selected from the amplicon pool if the unknown base N is identical to the assumed base b.

11. The system according to claim 10, wherein the P(S|N=b) is calculated by a method comprising:
defining a fraction of false bases F_{R}, wherein the fraction of false bases F_{R} is a ratio of an amplified false base to the amplified bases in the amplicon pool, and the amplified false base is a base distinct from the assumed base b; and
calculating the P(S|N=b) through the following equation:
$$P ( S \vert N = b ) = {\sum }_{{F}_{R}} P ( {F}_{R} ) \cdot P ( S \vert N = {b∩F}_{R} = {F}_{R} ) ;$$
wherein the P(S|N=b ∩F_{R}=F_{R}) is a sum product of P(sⱼ|N=b ∩F_{R}=F_{R}) of each observed base sⱼ if the unknown base N is identical to the assumed base b and a ratio of the amplified false base in the amplicon pool is F_{R}.

12. The system according to claim 11, wherein:
if the observed base sⱼ is identical to the assumed base b, the P(sⱼ|N=b ∩F_{R}=F_{R}) is equal to a fraction of true bases F_{R}′;
if the observed base sⱼ is distinct from the assumed base b, the P(sⱼ|N=b ∩F_{R}=F_{R}) is equal to the fraction of false bases F_{R}, and wherein a sum of the fraction of true bases F_{R}′ and the fraction of false bases F_{R} is 1.

13. The system according to claim 12, further comprising a consensus base error rate P_{c(S)} modifying module configured in the polymerase chain reaction quality evaluation module, and being signally connected to the sequencing quality evaluation module so as to modify the P(sⱼ|N=b ∩F_{R}=F_{R}) to be P(sⱼ=b′|N=b ∩{F_{b″≠b}}={F_{b″≠b}}) based on the sequencing accuracy rate P_{seq1}, the sequencing error rate P_{seq2} and an amplified base ratio F_{b″≠b}, where the P(sⱼ=b′|N=b ∩{F_{b″≠b}}={F_{b″≠b}}) represents a probability that the observed base sⱼ is identical to the another observed base b′ if the unknown base N is identical to the assumed base b, and a ratio of a amplified base b″ distinct from the assumed base b in the amplicon pool is F_{b″≠b}, and wherein:
if the amplified base b″ is distinct from the assumed base b, and the ratio of the amplified base F_{b″≠b}, is equal to the fraction of amplified bases F_{R}, the P(sⱼ=b′|N=b ∩{F_{b″≠b}}{F_{b″b}}) is modified to satisfy the following equation:
$$P ( {s}_{j} = {b}^{′} \vert N = b ∩ \{ {F}_{{b}^{″} \neq b} \} = \{ {F}_{{b}^{″} \neq b} \} ) = \{ \begin{matrix}{P}_{pcr 1} \times {P}_{seq 1} + {P}_{pcr 2} \times {P}_{seq 2} & {b}^{′} = b \\ {P}_{pcr 3} \times {P}_{seq 1} + {P}_{pcr 4} \times {P}_{seq 2} + {P}_{pcr 5} \times {P}_{seq 2} & {b}^{′} \neq b\end{matrix} ;$$
wherein, if the another observed base b′ is identical to the assumed base b, Pₚₑᵣ₁ represents a probability that the assumed base b is amplified into a base identical to the another observed base b′, and Pₚₑᵣ₂ represents a probability that the assumed base b is amplified into the amplified base b″, where the amplified base b″ is distinct from the assumed base b;
wherein, if the another observed base b′ is distinct from the assumed base b, Pₚₑᵣ₃ represents a probability that the assumed base b is amplified into the another observed base b′, Pₚₑᵣ₄ represents a probability that the assumed base b is amplified into a base identical to the assumed base b, and Pₚₑᵣ₅ represents a probability that the assumed base b is amplified into the amplified base b″, where the amplified base b″ is distinct from the assumed base b and also distinct from the another observed base b′;
wherein, the sequencing accuracy rate P_{seq1} represents a probability that the another observed base b′ is correctly sequenced, and the sequencing error rate P_{seq2} represents a probability that the amplified base b″ is falsely sequenced as the another observed base b′.

14. The system according to claim 13, further comprising a sequencing quality evaluation module, being signally connected to the polymerase chain reaction quality evaluation module, and configured to read a sequencing quality score Qᵢ of another observed base b′ so as to calculate the sequencing accuracy rate P_{seq1} and the sequencing error rate P_{seq2}, wherein:
the sequencing accuracy rate P_{seq1} is calculated through the following equation:
$${P}_{seq 1} = 1 - 1 {0}^{- {Q}_{i} / 10} ;$$
the sequencing error rate P_{seq2} is calculated through the following equation:
$${P}_{seq 2} = \frac{{10}^{- {Q}_{i} / 10}}{3} .$$

15. The system according to claim 12, wherein the observed base collection S comprises d observed bases sⱼ, wherein d observed bases sⱼ comprise n_{b} bases identical to the assumed base b and (d-nb) the amplified false bases distinct from the assumed base b, and wherein the d is the maximum integer value of j, and n_{b} is a positive integer less than or equal to d;
if the observed base sⱼ is distinct from the assumed base b, the P(sⱼ|N=b ∩F_{R}=F_{R}) is equal to the fraction of false bases F_{R}, and a sum product of the P(sⱼ|N=b ∩F_{R}=F_{R}) is calculated through the following equation:
$${\prod }_{j = 1}^{d} P ( {s}_{j} \vert N = b ∩ {F}_{R} = {F}_{R} ) = {( 1 - {F}_{R} )}^{{n}_{b}} {F}_{R}^{d - {n}_{b}} .$$

16. The system according to claim 10, wherein the P(S|N=b) is obtained by moment approximation through the following equation:
$$P ( S \vert N = b ) = {\sum }_{k = 0}^{{n}_{b}} {{C}_{k}^{{n}_{b}} ( - 1 )}^{k} {M}_{( k + d - {n}_{b} )} ;$$
wherein the M_{(k+d-n}_{_{b}}₎ is a (k+d-n_{b})^{th} moment among a probability distribution of the fraction of false bases F_{R}, where k is any one of non-negative integer less than or equal to n_{b};
wherein an approximated value of the M_{(k+d-n}_{_{b}}₎ is r/2^{k+d-nb}, where r represents a probability of polymerase chain reaction error occurrence at a gene locus corresponding to the unknown base N in each cycle.

17. The system according to claim 9, further comprises:
a scoring module, being signally connected to the polymerase chain reaction quality evaluation module, configured to identify the consensus base error rate P_{c(S)} and assign the consensus base c(S) a quality score Q_{c}; and
a determining module, being signally connected to the scoring module and the polymerase chain reaction quality evaluation module, configured to receive the quality score Q_{c} and compare the quality score Q_{c} with a second quality score P′_{c(S)} so as to generate a determined instruct, wherein the second quality score P′_{c(S)} is output by the polymerase chain reaction quality evaluation module, where:
if the second quality score P′_{c(S)} larger than the quality score Q_{c}, the determined instruct presents that the consensus base c(S) is confident;
if the second quality score P′_{c(S)} smaller than the quality score Q_{c}, the determined instruct presents that the consensus base c(S) is not confident.

18. A device for evaluating consensus base error rate, comprising a memory and a processor, wherein the memory is configured to store a program, and the processor is configured to execute the program so as to realize the system according to claim 9.

19. The device according to claim 18, wherein the system further comprises a consensus base error rate P_{c(S)} modifying module configured in the polymerase chain reaction quality evaluation module, and being signally connected to the sequencing quality evaluation module so as to modify the P(sⱼ|N=b ∩F_{R}=F_{R}) to be P(sⱼ=b′|N=b ∩{F_{b″≠b}}={F_{b″≠b}}) based on the sequencing accuracy rate P_{seq1}, the sequencing error rate P_{seq2} and an amplified base ratio F_{b″≠b}, where the P(sⱼ=b′|N=b ∩{F_{b″≠b}}={F_{b″≠b}}) represents a probability that the observed base sⱼ is identical to the another observed base b′ if the unknown base N is identical to the assumed base b, and a ratio of a amplified base b″ distinct from the assumed base b in the amplicon pool is F_{b″≠b}, and wherein:
if the amplified base b″ is distinct from the assumed base b, and the ratio of the amplified base F_{b″≠b} is equal to the fraction of amplified bases F_{R}, the P(sⱼ=b′|N=b ∩{F_{b″≠b}}={F_{b″≠b}}) is modified to satisfy the following equation:
$$P ( {s}_{j} = {b}^{′} \vert N = b ∩ \{ {F}_{{b}^{″} \neq b} \} = \{ {F}_{{b}^{″} \neq b} \} ) = \{ \begin{matrix}{P}_{pcr 1} \times {P}_{seq 1} + {P}_{pcr 2} \times {P}_{seq 2} & {b}^{′} = b \\ {P}_{pcr 3} \times {P}_{seq 1} + {P}_{pcr 4} \times {P}_{seq 2} + {P}_{pcr 5} \times {P}_{seq 2} & {b}^{′} \neq b\end{matrix} ;$$
wherein, if the another observed base b′ is identical to the assumed base b, Pₚₑᵣ₁ represents a probability that the assumed base b is amplified into a base identical to the another observed base b′, and Pₚₑᵣ₂ represents a probability that the assumed base b is amplified into the amplified base b″, where the amplified base b″ is distinct from the assumed base b;
wherein, if the another observed base b′ is distinct from the assumed base b, Pₚₑᵣ₃ represents a probability that the assumed base b is amplified into the another observed base b′, Pₚₑᵣ₄ represents a probability that the assumed base b is amplified into a base identical to the assumed base b, and Pₚₑᵣ₅ represents a probability that the assumed base b is amplified into the amplified base b″, where the amplified base b″ is distinct from the assumed base b and also distinct from the another observed base b′;
wherein, the sequencing accuracy rate P_{seq1} represents a probability that the another observed base b′ is correctly sequenced, and the sequencing error rate P_{seq2} represents a probability that the amplified base b″ is falsely sequenced as the another observed base b′.

20. The device according to claim 19, wherein the system further comprises a sequencing quality evaluation module, being signally connected to the polymerase chain reaction quality evaluation module, and configured to read a sequencing quality score Qᵢ of another observed base b′ so as to calculate the sequencing accuracy rate P_{seq1} and the sequencing error rate P_{seq2}, wherein:
the sequencing accuracy rate P_{seq1} is calculated through the following equation:
$${P}_{seq 1} = 1 - 1 {0}^{- {Q}_{i} / 10} ;$$
the sequencing error rate P_{seq2} is calculated through the following equation:
$${P}_{seq 2} = \frac{1 {0}^{- {Q}_{i} / 10}}{3} .$$
